(19) United States
(12) Patent Application Publication
(10) Pub. No.: US 2003/0108934 A1
(43) Pub. Date: Jun. 12, 2003

(54) 21910, a novel human membrane-associated guanylate kinase and uses thereof

(75) Inventors: Rosana Kapeller-Libermann, Chestnut Hill, MA (US); John Joseph Hunter, Somerville, MA (US)

Correspondence Address: LAHIVE & COCKFIELD, 28 STATE STREET, BOSTON, MA, 02109, US

(73) Assignee: Millennium Pharmaceuticals, Inc., Cambridge, MA (US)

(21) Appl. No.: 10/278,036

(22) Filed: Oct. 22, 2002

Related U.S. Application Data
(63) Continuation of application No. 09/711,216, filed on Nov. 9, 2000, now abandoned.
(60) Provisional application No. 60/205,447, filed on May 19, 2000.

(51) Int. Cl. C12Q 1/68; G01N 33/574; C07H 21/04; C12N 9/12; C12P 21/02; C12N 5/06

(52) U.S. Cl. 435/6; 435/7.23; 435/69.1; 435/194; 435/320.1; 435/325; 530/388.26; 536/23.2

(57) ABSTRACT

The invention provides isolated nucleic acid molecules, designated MAGK nucleic acid molecules, which encode novel guanylate kinase related molecules. The invention also provides antisense nucleic acid molecules, recombinant expression vectors containing MAGK nucleic acid molecules, host cells into which the expression vectors have been introduced, and nonhuman transgenic animals in which a MAGK gene has been introduced or disrupted. The invention still further provides isolated MAGK proteins, fusion proteins, antigenic peptides and anti-MAGK antibodies. Treatment and diagnostic methods for cellular growth or proliferation diseases or disorders, e.g., cancer, including, but not limited to colon cancer and lung cancer, utilizing compositions of the invention, are also provided.

### RELATED APPLICATIONS

This application claims the benefit of prior-filed provisional patent application Serial No. 60/205,447, filed May 19, 2000, entitled “21910, A Novel Human Membrane-Associated Guanylate Kinase and Uses Thereof”. The entire content of the above-referenced application is incorporated herein by this reference.

### BACKGROUND OF THE INVENTION

Guanylate kinases are essential enzymes in nucleotide metabolism pathways catalyzing the ATP-dependent phosphorylation of either GMP to GDP or dGMP to dGDP. Guanyate kinase molecules also function in the recovery of cGMP (cGMP→GMP→GDP→GTP→cGMP) thereby serving to regulate the supply of guanine nucleotides to signal transduction pathway components (Brady et al. (1996) J. Biol. Chem. 271(28):16734-40; Kumar, et al. (2000) Eur. J. Biochem. 267(2):606). Guanylate kinases are essential to a wide range of cellular processes including but not limited to nucleotide metabolic processes (e.g., supplying the building blocks for nucleic acids), phototransduction processes (e.g., regulating the opening and/or closing of cGMP gated-channels), cellular growth and proliferation, and signaling pathways (Fitzgibbon, et al (1996) FEBS Letters 385:185-188).

Membrane-bound forms of guanylate kinase molecules have also been discovered. Members of the membrane-associated guanylate kinase family interact with the cytoskeleton of the cell and regulate cell proliferation, signaling pathways, and intercellular junctions. (Kim, et al. (1996) Genomics 31(2):223). These molecules participate in the assembly of multiprotein complexes on the inner surface of the plasma membrane and cluster ion channels, receptors, adhesion molecules and cytosolic signaling proteins at synapses, cellular junctions, and polarized membrane domains (Fannin and Anderson (1999) Curr. Opin. Cell Biol. 11(4):432; Dobrosotskaya, et al. (1997) J. Biol. Chem. 272(50):31589). In addition, membrane-associated guanylate kinases have recently been found to have a transcriptional regulatory function (Hsueh, et al. (2000) Nature 404(6775):298). Typically, these molecules contain multiple protein-protein interaction motifs including a PDZ domain in the N-terminal portion of the protein, followed by a SH3 domain, followed by a guanylate kinase domain at the C-terminus (Dobrosotskaya, et al., supra). Membrane-associated guanylate kinases have been found to be localized to tight junctions in epithelial cell membranes and more notably in neuronal cells (Wu, et al. (2000) Proc. Natl Acad. Sci. USA 97(8):4233); Hsuesh, supra).

In humans, guanylate kinases are used as targets for cancer chemotherapy and have been found to be inhibited by the antitumor drug, 6-thioguanine. In addition, guanylate kinase activity is required for the activation of antiviral drugs such as acyclovir and ganciclovir in virus-infected cells (Brady et al., supra).

Members of the guanylate kinase family have been identified in many organisms, including E. coli, yeast, mouse, and human. Greater conservation has been found between mammalian guanylate kinases than between mammalian and yeast or E. coli. However, the overall structure of the molecule is conserved, including conservation of a “giant anion hole” active site which functions to bind nucleoside triphosphates (Brady et al., supra; Stehle and Schulz (1992) J. Mol. Biol. 224(4):1127).

Given the wide range of important cellular processes in which guanylate kinases play an important role, there exists a need for identifying novel guanylate kinases as well as for identifying modulators for use in a variety of processes.

### SUMMARY OF THE INVENTION

The present invention is based, at least in part, on the discovery of novel members of the family of guanylate kinases (e.g. membrane-associated guanylate kinases) referred to herein as MAGK nucleic acid and protein molecules (e.g. membrane-associated guanylate kinases). The present invention is based, also in part, on the discovery that the novel MAGK molecules of the present invention have increased expression in tumor cells, e.g., lung tumor cells and colon tumor cells, as compared to normal lung and colon cells, and are useful in the diagnosis and treatment of cellular growth and proliferation diseases and disorders, e.g., cancer, including, but not limited to, lung cancer and colon cancer.

The MAGK nucleic acid and protein molecules of the present invention are useful as modulating agents in regulating a variety of cellular processes, e.g., nucleotide metabolism, cellular growth and proliferation, and cellular signaling e.g., cellular growth signaling. Accordingly, in one aspect, this invention provides isolated nucleic acid molecules encoding MAGK proteins or biologically active portions thereof, as well as nucleic acid fragments suitable as primers or hybridization probes for the detection of MAGK-encoding nucleic acids.

In one embodiment, a MAGK nucleic acid molecule of the invention is at least 50%, 55%, 60%, 65%, 70%, 75%, 80%, 85%, 90%, 91%, 92%, 93%, 94%, 95%, 96%, 97%, 98%, 99% or more identical to the nucleotide sequence (e.g., to the entire length of the nucleotide sequence) shown in SEQ ID NO:1 or 3, or the nucleotide sequence of the DNA insert of the plasmid deposited with ATCC as Accession Number ______ , or a complement thereof.

In a preferred embodiment, the isolated nucleic acid molecule includes the nucleotide sequence shown in SEQ ID NO:1 or 3, or a complement thereof. In another embodiment, the nucleic acid molecule includes SEQ ID NO:3 and nucleotides 1-451 of SEQ ID NO:1. In yet a further embodiment, the nucleic acid molecule includes SEQ ID NO:3 and nucleotides 2410-4281 of SEQ ID NO:1. In another preferred embodiment, the nucleic acid molecule consists of the nucleotide sequence shown in SEQ ID NO:1 or 3.

In another embodiment, a MAGK nucleic acid molecule includes a nucleotide sequence encoding a protein having an amino acid sequence sufficiently identical to the amino acid sequence of SEQ ID NO:2, or an amino acid sequence encoded by the DNA insert of the plasmid deposited with ATCC as Accession Number ______. In a preferred embodiment, a MAGK nucleic acid molecule includes a nucleotide sequence encoding a protein having an amino acid sequence at least 50%, 55%, 60%, 65%, 70%, 75%, 80%, 85%, 90%, 91%, 92%, 93%, 94%, 95%, 96%, 97%, 98%, 99% or more identical to the entire length of the amino acid sequence of SEQ ID NO:2, or the amino acid sequence encoded by the DNA insert of the plasmid deposited with ATCC as Accession Number ______.

In another preferred embodiment, an isolated nucleic acid molecule encodes the amino acid sequence of human MAGK. In yet another preferred embodiment, the nucleic acid molecule includes a nucleotide sequence encoding a protein having the amino acid sequence of SEQ ID NO:2, or the amino acid sequence encoded by the DNA insert of the plasmid deposited with ATCC as Accession Number ______. In yet another preferred embodiment, the nucleic acid molecule is at least 50-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300 or more nucleotides in length. In a further preferred embodiment, the nucleic acid molecule is at least 50-100, 100-200, 200-300, 300-400, 400-500, 500-600, 600-700, 700-800, 800-900-1000, 1000-1100, 1100-1200, 1200-1300, 1300-1400, 1400-1500, 1500-1600, 1600-1700, 1700-1800, 1800-1900, 1900-2000, 2000-2100,2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300 or more nucleotides in length and encodes a protein having a MAGK activity (as described herein).

Another embodiment of the invention features nucleic acid molecules, preferably MAGK nucleic acid molecules, which specifically detect MAGK nucleic acid molecules relative to nucleic acid molecules encoding non-MAGK proteins. For example, in one embodiment, such a nucleic acid molecule is at least 50-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300 or more nucleotides in length and hybridizes under stringent conditions to a nucleic acid molecule comprising the nucleotide sequence shown in SEQ ID NO:1, the nucleotide sequence of the DNA insert of the plasmid deposited with ATCC as Accession Number ______, or a complement thereof.

In preferred embodiments, the nucleic acid molecules are at least 15 (e.g., 15 contiguous) nucleotides in length and hybridize under stringent conditions to the nucleotide molecule set forth in SEQ ID NO:1.

In other preferred embodiments, the nucleic acid molecule encodes a naturally occurring allelic variant of a polypeptide comprising the amino acid sequence of SEQ ID NO:2, or an amino acid sequence encoded by the DNA insert of the plasmid deposited with ATCC as Accession Number ______, wherein the nucleic acid molecule hybridizes to a nucleic acid molecule comprising SEQ ID NO:1 or 3, respectively, under stringent conditions.

Another embodiment of the invention provides an isolated nucleic acid molecule which is antisense to a MAGK nucleic acid molecule, e.g., the coding strand of a MAGK nucleic acid molecule.

Another aspect of the invention provides a vector comprising a MAGK nucleic acid molecule. In certain embodiments, the vector is a recombinant expression vector. In another embodiment, the invention provides a host cell containing a vector of the invention. In yet another embodiment, the invention provides a host cell containing a nucleic acid molecule of the invention. The invention also provides a method for producing a protein, preferably a MAGK protein, by culturing in a suitable medium, a host cell, e.g., a mammalian host cell such as a non-human mammalian cell, of the invention containing a recombinant expression vector, such that the protein is produced.

Another aspect of this invention features isolated or recombinant MAGK proteins and polypeptides. In one embodiment, an isolated MAGK protein includes at least one or more of the following motifs or domains: an ATP/GTP-binding site motif A (P-loop), a guanylate kinase domain, a PDZ domain, and a SH3 domain.

In a preferred embodiment, a MAGK protein includes at least one or more of the following motifs or domains: an ATP/GTP-binding site motif A (P-loop), a guanylate kinase domain, a PDZ domain, a SH3 domain and has an amino acid sequence at least about 50%, 55%, 60%, 65%, 67%, 68%, 70%, 75%, 80%, 85%, 90%, 91%, 92%, 93%, 94%, 95%, 96%, 97%, 98%, 99% or more identical to the amino acid sequence of SEQ ID NO:2, or the amino acid sequence encoded by the DNA insert of the plasmid deposited with ATCC as Accession Number ______.

In another preferred embodiment, a MAGK protein includes at least one or more of the following motifs or domains: an ATP/GTP-binding site motif A (P-loop), a guanylate kinase domain, a PDZ domain, a SH3 domain and has a MAGK activity (as described herein).

In yet another preferred embodiment, a MAGK protein includes at least one or more of the following motifs or domains: an ATP/GTP-binding site motif A (P-loop), a guanylate kinase domain, a PDZ domain, a SH3 domain and is encoded by a nucleic acid molecule having a nucleotide sequence which hybridizes under stringent hybridization conditions to a complement of a nucleic acid molecule comprising the nucleotide sequence of SEQ ID NO:1 or 3.

In another embodiment, the invention features fragments of the protein having the amino acid sequence of SEQ ID NO:2, wherein the fragment comprises at least 15 amino acids (e.g., contiguous amino acids) of the amino acid sequence of SEQ ID NO:2, or an amino acid sequence encoded by the DNA insert of the plasmid deposited with the ATCC as Accession Number ______. In another embodiment, a MAGK protein has the amino acid sequence of SEQ ID NO:2.

In another embodiment, the invention features a MAGK protein which is encoded by a nucleic acid molecule consisting of a nucleotide sequence at least about 50%, 55%, 60%, 65%, 70%, 75%, 80%, 85%, 90%, 91%, 92%, 93%, 94%, 95%, 96%, 97%, 98%, 99% or more identical to a nucleotide sequence of SEQ ID NO:1 or 3, or a complement thereof. This invention further features a MAGK protein which is encoded by a nucleic acid molecule consisting of a nucleotide sequence which hybridizes under stringent hybridization conditions to a complement of a nucleic acid molecule comprising the nucleotide sequence of SEQ ID NO:1 or 3.

The proteins of the present invention or portions thereof, e.g., biologically active portions thereof, can be operatively linked to a non-MAGK polypeptide (e.g., heterologous amino acid sequences) to form fusion proteins. The invention further features antibodies, such as monoclonal or polyclonal antibodies, that specifically bind proteins of the invention, preferably MAGK proteins. In addition, the MAGK proteins or biologically active portions thereof can be incorporated into pharmaceutical compositions, which optionally include pharmaceutically acceptable carriers.

In another aspect, the present invention provides a method for detecting the presence of a MAGK nucleic acid molecule, protein, or polypeptide in a biological sample, e.g., a lung or colon tissue sample or tumor sample, by contacting the biological sample with an agent capable of detecting a MAGK nucleic acid molecule, protein, or polypeptide such that the presence of a MAGK nucleic acid molecule, protein or polypeptide is detected in the biological sample. In one embodiment, the sample comprises mRNA molecules and is contacted with a nucleic acid probe.

In another aspect, the present invention provides a method for detecting the presence of MAGK activity in a biological sample by contacting the biological sample with an agent capable of detecting an indicator of MAGK activity such that the presence of MAGK activity is detected in the biological sample, e.g., a lung or colon tissue sample or tumor sample.

In another aspect, the invention provides a method for modulating MAGK activity comprising contacting a cell capable of expressing MAGK with an agent that modulates MAGK activity such that MAGK activity in the cell is modulated. In one embodiment, the agent inhibits MAGK activity. In another embodiment, the agent stimulates MAGK activity. In one embodiment, the agent is an antibody that specifically binds to a MAGK protein. In another embodiment, the agent modulates expression of MAGK by modulating transcription of a MAGK gene or translation of a MAGK mRNA. In yet another embodiment, the agent is a nucleic acid molecule having a nucleotide sequence that is antisense to the coding strand of a MAGK mRNA or a MAGK gene.

In another embodiment, the methods of the present invention are used to treat a subject having a disorder characterized by aberrant or unwanted MAGK protein or nucleic acid expression or activity, e.g., a cellular growth or proliferation disease or disorder such as cancer, by administering an agent which is a MAGK modulator to the subject. In one embodiment, the MAGK modulator is a MAGK protein. In another embodiment the MAGK modulator is a MAGK nucleic acid molecule, an antisense MAGK nucleic acid molecule, or a ribozyme. In yet another embodiment, the MAGK modulator is a peptide, peptidomimetic, or other small molecule. In a preferred embodiment, the disorder characterized by aberrant or unwanted MAGK protein or nucleic acid expression is a membrane-associated guanylate kinase-associated disorder, e.g., a cellular growth or proliferation disease or disorder such as, for example, colon cancer or lung cancer.

The present invention also provides diagnostic assays for identifying the presence or absence of a genetic alteration characterized by at least one of (i) aberrant modification or mutation of a gene encoding a MAGK protein; (ii) mis-regulation of the gene, e.g., overexpression of the gene; and (iii) aberrant post-translational modification of a MAGK protein, wherein a wild-type form of the gene encodes a protein with a MAGK activity.

In another aspect the invention provides methods for identifying a compound that binds to or modulates the activity of a MAGK protein, by providing an indicator composition comprising a MAGK protein having MAGK activity, contacting the indicator composition with a test compound, and determining the effect of the test compound on MAGK activity in the indicator composition to identify a compound that modulates the activity of a MAGK protein.

In yet another aspect, the invention provides a method for identifying a compound capable of modulating cell proliferation, cell growth, or cell signaling, by contacting a cell with a test compound and assaying the ability of the test compound to modulate the expression of a MAGK nucleic acid or the activity of an MAGK polypeptide.

Also featured are methods of regulating metastasis in an individual or inhibiting tumor progression in an individual which include administering to the individual a MAGK modulator (e.g., a MAGK inhibitor).

Other features and advantages of the invention will be apparent from the following detailed description and claims.

### BRIEF DESCRIPTION OF THE DRAWINGS

FIGS. 1A-C depicts the cDNA sequence and predicted amino acid sequence of human MAGK (clone Fbh21910). The nucleotide sequence corresponds to nucleic acids 1 to 4281 of SEQ ID NO:1. The amino acid sequence corresponds to amino acids 1 to 675 of SEQ ID NO:2. The coding region without the 3′ untranslated region of the human MAGK gene is shown in SEQ ID NO:3.

FIG. 2 depicts a hydrophobicity analysis of the human MAGK protein.

FIGS. 3A-C depicts the results of a search which was performed against the HMM database and which resulted in the identification of a “guanylate kinase domain”, a “PDZ domain”, and a “SH3 domain” in the human MAGK protein.

FIGS. 4A-B depicts a multiple sequence alignment (MSA) of the amino acid sequences of the human MAGK protein (SEQ ID NO:2), the CG1617 gene product from Drosophila melanogaster (GenBank Accession No. AAF46351), and p55-related membrane associated guanylate kinase protein DLG3 (GenBank Accession No. AAD39392). The alignment was performed using the Clustal algorithm which is part of the MegAlign™ program (e.g., version 3.1.7), which is part of the DNAStar™ sequence analysis software package. The pairwise alignment parameters are as follows: K-tuple=1; Gap Penalty=3; Window=5; Diagonals saved =5 . The multiple alignment parameters are as follows: Gap Penalty=10; and Gap length penalty=10.

FIG. 5 is a graphic depiction of the relative levels of the human MAGK mRNA in a small cell lung carcinoma cell line (NHBE) as compared to normal bronchial epithelium (NCI-H69).

### DETAILED DESCRIPTION OF THE INVENTION

The present invention is based, at least in part, on the discovery of novel molecules, referred to herein as “membrane-associated guanylate kinase” or “MAGK” nucleic acid and protein molecules. Guanylate kinase molecules are novel members of a family of enzymes possessing kinase activity. These novel molecules are capable of catalyzing the ATP-dependent phosphorylation of GMP to GDP or dGMP to dGDP and function in the recovery of cGMP. Membrane-associated guanylate kinases are capable of interaction with the cytoskeleton of the cell and are capable of participating in the assembly of multiprotein complexes. The novel MAGK molecules of the invention may thus play a role in or function in a variety of cellular processes, e.g., cellular proliferation, cellular signaling, growth, differentiation, migration, and inter- or intra-cellular communication.

The MAGK molecules of the present invention, through association with cell surface signaling complexes involved in cellular growth and proliferation, may play a role in the modulation of cellular growth signaling mechanisms. As used herein, the terms “cellular growth signaling mechanisms,” “cell signaling,” or “cell growth signaling” includes signal transmission from a cell surface signaling complex which regulates, for example, 1) cell transversal through the cell cycle, 2) cell differentiation, 3) cell survival, and/or 4) cell migration.

In a preferred embodiment, the MAGK molecules of the present invention are involved in metabolic processes of the cell and in the modulation of cellular growth signaling mechanisms. Thus, the MAGK molecules may modulate cellular growth, differentiation, or migration, and may play a role in disorders characterized by aberrantly regulated growth, proliferation, differentiation, or migration. Accordingly, in one aspect, the present invention provides methods and compositions for the diagnosis and treatment of a cellular growth or proliferation disease or disorder, e.g., cancer, including, but not limited to, lung cancer and colon cancer.

The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

A “cellular growth or proliferation disease or disorder” includes those diseases or disorders that affect cell growth or proliferation processes. As used herein, a “cellular growth or proliferation process” is a process by which a cell increases in number, size or content, by which a cell develops a specialized set of characteristics which differ from that of other cells, or by which a cell moves closer to or further from a particular location or stimulus. Such disorders include, but are not limited to, cancer, e.g., carcinoma, sarcoma, or leukemia, examples of which include, but are not limited to, colon, lung, liver, ovary, and breast; tumorigenesis and metastasis; skeletal dysplasia; hepatic disorders; and hematopoietic and/or myeloproliferative disorders.

The novel MAGK molecules of the present invention have increased expression in tumor cells, e.g., lung tumor cells and colon tumor cells, as compared to normal lung and colon cells. Increased expression of MAGK in tumor cells results in an increase in cell growth signaling, thereby increasing the cellular growth and proliferation of tumor cells. Accordingly, the MAGK molecules of the present invention provide novel diagnostic targets and therapeutic agents to control MAGK-related disorders, e.g., cellular growth or proliferation diseases or disorders, e.g., cancer, including, but not limited to colon cancer or lung cancer. Accordingly, the present invention further provides methods for identifying the presence of a MAGK nucleic acid or polypeptide molecule associated with a cellular growth or proliferation disease or disorder. In addition, the invention provides methods for identifying a subject at risk for a cellular growth or proliferation disease or disorder, by detecting the presence of a MAGK nucleic acid or polypeptide molecule, or by detecting aberrant or abnormal MAGK expression or activity.

The invention also provides a method for identifying a compound capable of treating a cellular growth or proliferation disease or disorder, characterized by aberrant MAGK nucleic acid expression or MAGK protein activity by assaying the ability of the compound to modulate the expression of a MAGK nucleic acid or the activity of a MAGK protein. Furthermore, the invention provides a method for treating a subject having a cellular growth or proliferation disease or disorder characterized by aberrant MAGK protein activity or aberrant MAGK nucleic acid expression by administering to the subject a MAGK modulator which is capable of modulating MAGK protein activity or MAGK nucleic acid expression.

Moreover, the invention provides a method for identifying a compound capable of modulating cellular growth and/or proliferation and cellular signaling by modulating the expression of a MAGK nucleic acid or the activity of a MAGK protein. The invention provides a method for modulating cellular growth and/or proliferation and cellular signaling comprising contacting an endothelial cell with a MAGK modulator.

The present invention is directed to novel members of the guanylate kinase family of enzymes, e.g. the MAGK proteins, biologically active fragments thereof, homologues thereof, and/or nucleic acid molecules encoding such proteins, homologues and/or biologically active fragments, and the use thereof for treating and/or diagnosing a cellular growth or proliferation disease or disorder. The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin, e.g., mouse or monkey proteins. Members of a family may also have common functional characteristics.

Accordingly, in one embodiment, a MAGK molecule of the present invention is identified based on the presence of a “ATP/GTP-binding site motif A (P-loop)” in the protein or corresponding nucleic acid molecule. As used herein, the term “ATP/GTP-binding site motif A (P-loop)” includes a protein motif having an amino acid sequence of about 8 amino acid residues. Preferably, a P-loop has about 5-8 residues and the following consensus sequence: [AG]-X(4)-G-K-[ST] (Saraste M., Sibbald P. R., Wittinghofer A. (1990) Trends Biochem. Sci. 15:430-434). To identify the presence of a ATP/GTP-binding site motif A (P-loop) in a MAGK protein, and make the determination that a protein of interest has a particular motif, the amino acid sequence of the protein may be searched against a database of known protein motifs (e.g., the ProSite database). The ATP/GTP-binding site motif A (P-loop) has been assigned ProSite accession number PS00017 (http://www.expasy.ch/cgi-bin/prosite-search-ac?PS00017). A search was performed against the ProSite database resulting in the identification of a ATP/GTP-binding site motif A (P-loop) in the amino acid sequence of human MAGK (SEQ ID NO:2) at about residues 404-411 of SEQ ID NO:2.

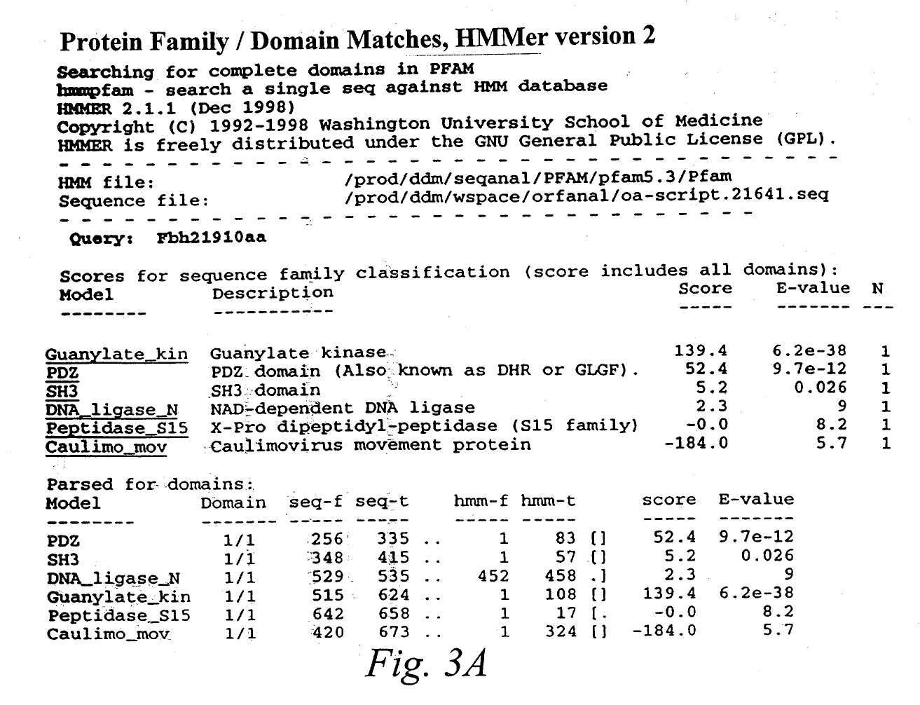

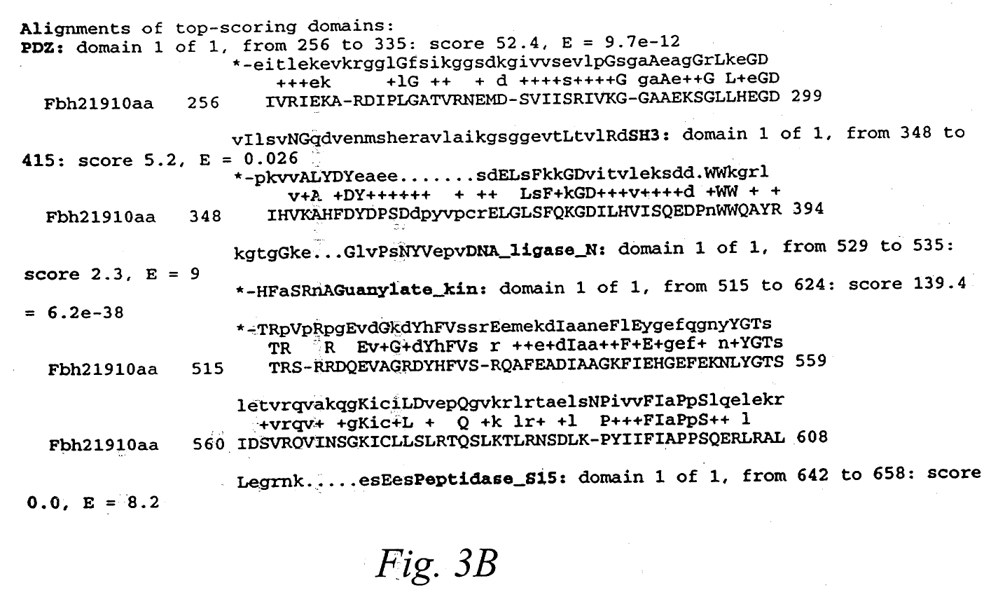

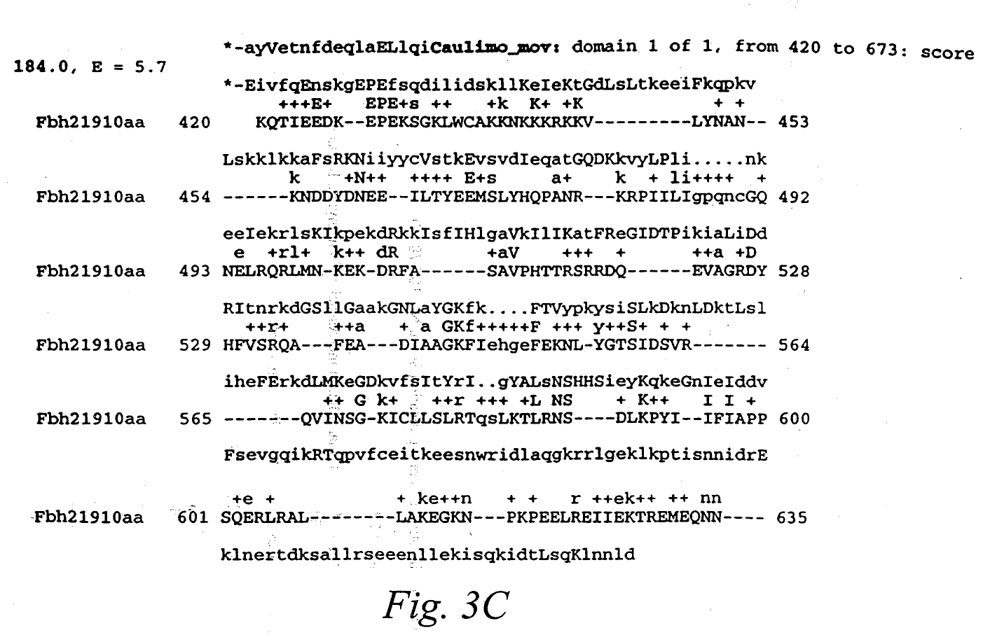

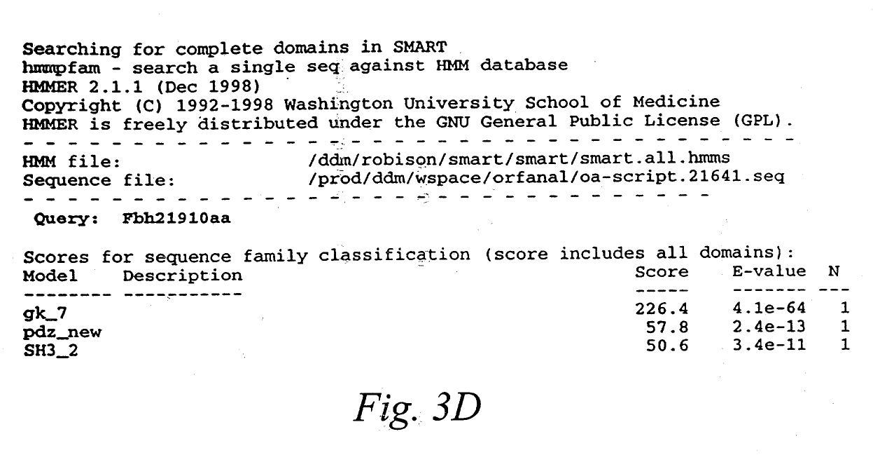

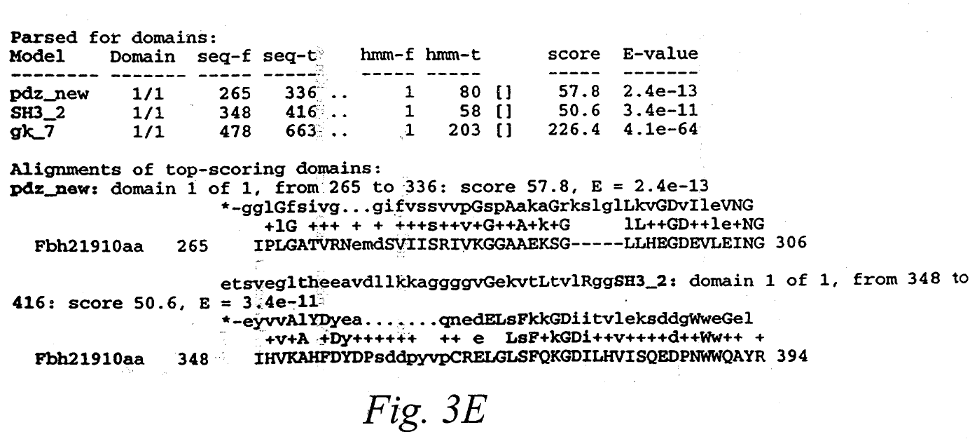

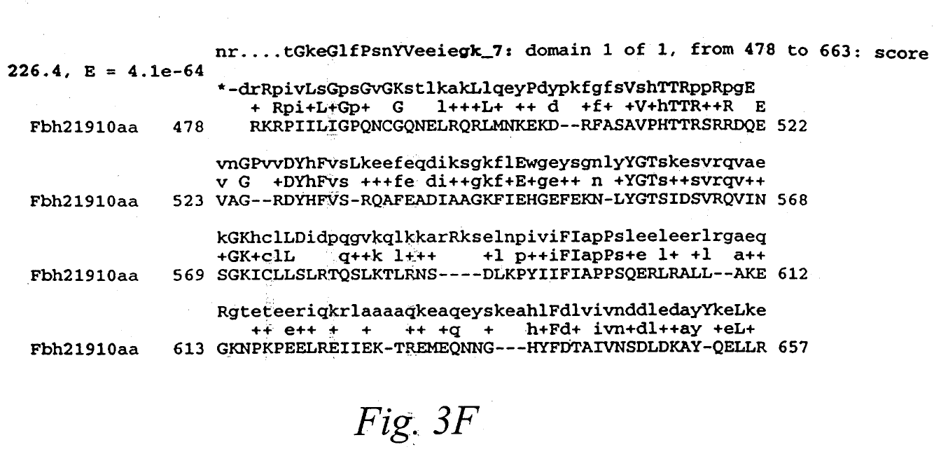

In another embodiment, a MAGK molecule of the present invention is identified based on the presence of a “guanylate kinase domain” in the protein or corresponding nucleic acid molecule. As used herein, the term “guanylate kinase domain” includes a protein domain having an amino acid sequence of about 50-200 amino acid residues and a bit score of about 50, 60, 70, 80, 90, 100, 110, 120, 130, 140, 150, 160, 170, 180, 190, 200, 210, or 220 or more. Preferably, a guanylate kinase domain includes at least about 100-200, or more preferably about 109 amino acid residues, and a bit score of at least 139.4. To identify the presence of a guanylate kinase domain in a MAGK protein, and make the determination that a protein of interest has a particular profile, the amino acid sequence of the protein may be searched against a database of known protein domains (e.g., the HMM database). A search was performed against the HMM database resulting in the identification of a guanylate kinase domain in the amino acid sequence of human MAGK (SEQ ID NO:2) at about residues 515-624 of SEQ ID NO:2. The results of the search are set forth in FIGS. 3A-C.

A guanylate kinase domain can further be characterized based on the presence of a guanylate kinase consensus sequence in the protein or corresponding nucleic acid molecule. As used herein, the term “guanylate kinase domain” includes a protein motif having an amino acid sequence of about 18 amino acid residues. Preferably, a guanylate kinase domain has about 15-20 residues. To identify the presence of a guanylate kinase domain in a MAGK protein, and make the determination that a protein of interest has a particular motif, the amino acid sequence of the protein may be searched against a database of known protein motifs (e.g., the ProSite database). The guanylate kinase domain has been assigned ProSite accession number PS (http://www.expasy.ch/cgi-bin/prosite-search-ac?PS00856). A search was performed against the ProSite database resulting in the identification of a guanylate kinase domain in the amino acid sequence of human MAGK (SEQ ID NO:2) at about residues 514-531 of SEQ ID NO:2.

In another embodiment, a MAGK molecule of the present invention is identified based on the presence of a “PDZ domain” in the protein or corresponding nucleic acid molecule. As used herein, the term “PDZ domain” includes a protein domain having an amino acid sequence of about 50-200 amino acid residues and a bit score of about 20, 30, 40, 50, 60, 70, 80, 90, 100, 110, 120, 130, 140, 150, 160, 170, 180, 190 or 200 or more. Preferably, a PDZ domain includes at least about 50-150, or more preferably about 79 amino acid residues, and a bit score of at least 52.4. To identify the presence of a PDZ domain in a MAGK protein, and make the determination that a protein of interest has a particular profile, the amino acid sequence of the protein may be searched against a database of known protein domains (e.g., the HMM database). A search was performed against the HMM database resulting in the identification of a PDZ domain in the amino acid sequence of human MAGK (SEQ ID NO:2) at about residues 256-335 of SEQ ID NO:2. The results of the search are set forth in FIGS. 3A-C.

In another embodiment, a MAGK molecule of the present invention is identified based on the presence of a “SH3 domain” in the protein or corresponding nucleic acid molecule. As used herein, the term “SH3 domain” includes a protein domain having an amino acid sequence of about 50-150 amino acid residues and a bit score of about 5, 10, 20, 30, 40, 50, 60, 70, 80, 90, 100, 110, 120, 130, 140, 150 or more. Preferably, a SH3 domain includes at least about 50-100, or more preferably about 67 amino acid residues, and a bit score of at least 5.2. To identify the presence of a SH3 domain in a MAGK protein, and make the determination that a protein of interest has a particular profile, the amino acid sequence of the protein may be searched against a database of known protein domains (e.g., the HMM database). A search was performed against the HMM database resulting in the identification of a SH3 domain in the amino acid sequence of human MAGK (SEQ ID NO:2) at about residues 348-415 of SEQ ID NO:2. The results of the search are set forth in FIGS. 3A-C.

In a preferred embodiment, the MAGK molecules of the invention include at least one, preferably two, more preferably three or more or more of the following domains: an ATP/GTP-binding site motif A (P-loop), a guanylate kinase domain, a PDZ domain, and a SH3 domain.

In yet another embodiment, isolated proteins of the present invention, preferably MAGK proteins, have an amino acid sequence sufficiently identical to the amino acid sequence of SEQ ID NO:2, or are encoded by a nucleotide sequence sufficiently identical to SEQ ID NO:1 or 3. As used herein, the term “sufficiently identical” refers to a first amino acid or nucleotide sequence which contains a sufficient or minimum number of identical or equivalent (e.g, an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 30%, 40%, or 50% homology, preferably 60% homology, more preferably 70%-80%, and even more preferably 90-95% homology across the amino acid sequences of the domains and contain at least one and preferably two structural domains or motifs, are defined herein as sufficiently identical. Furthermore, amino acid or nucleotide sequences which share at least 30%, 40%, or 50%, preferably 60%, more preferably 70-80%, or 90-95% homology and share a common functional activity are defined herein as sufficiently identical.

As used interchangeably herein, an “MAGK activity”, “biological activity of MAGK,” or “functional activity of MAGK,” refers to an activity exerted by a MAGK protein, polypeptide or nucleic acid molecule on a MAGK responsive cell or tissue, or on a MAGK protein substrate, as determined in vivo, or in vitro, according to standard techniques. As used herein, a “membrane-associated guanylate kinase activity” includes ATP-dependent phosphorylation of GMP (or dGMP) into GDP (or dGDP) involved, for example, in the production of molecules necessary for signal transduction, cell signaling, cellular growth, cellular proliferation, and the like. In one embodiment, a MAGK activity is a direct activity, such as an association with a MAGK-target molecule. As used herein, a “target molecule” or “binding partner” is a molecule with which a MAGK protein binds or interacts in nature, such that MAGK-mediated function is achieved, e.g., modulation of cellular signaling, growth, and/or proliferation. A MAGK target molecule can be a non-MAGK molecule or a MAGK protein or polypeptide of the present invention (e.g., ATP). In an exemplary embodiment, a MAGK target molecule is a MAGK ligand (e.g., GMP, dGMP). Alternatively, a MAGK activity is an indirect activity, such as a cellular signaling activity mediated by interaction of the MAGK protein with a MAGK ligand. The biological activities of MAGK are described herein. For example, the MAGK proteins of the present invention can have one or more of the following activities: i) interaction of a MAGK protein molecule with a non-MAGK protein molecule (e.g. GMP, ATP), ii) modification of a MAGK substrate (e.g. GMP or dGMP), iii) assembly of protein complexes at cell-junctions, iv) interaction with the cellular cytoskeleton, and v) interaction between a membrane-bound MAGK protein and a non-MAGK protein. In yet another preferred embodiment, a MAGK activity is at least one or more of the following activities: 1) modulation of ATP-dependent phosphorylation of GMP, dGMP, or cGMP 2) modulation of cellular signal transduction, 3) modulation of metabolism or catabolism of metabolically important biomolecules (e.g., nucleotides), 4) modulation of cellular growth and differentiation, 5) modulation of cellular proliferation, a 6) modulation of cell signaling mechanisms, e.g., cellular growth signaling mechanisms, 7) modulation of intercellular junctions, 8) modulation of transcription, and 9) modulation of paracellular pathways.

Accordingly, another embodiment of the invention features isolated MAGK proteins and polypeptides having a MAGK activity. Other preferred proteins are MAGK proteins having one or more of the following domains: an ATP/GTP-binding site motif A (P-loop), a guanylate kinase domain, a PDZ domain, a SH3 domain, and, preferably, a MAGK activity.

Additional preferred proteins have one or more of the following domains: an ATP/GTP-binding site motif A (P-loop), a guanylate kinase domain, a PDZ domain, a SH3 domain, and are, preferably, encoded by a nucleic acid molecule having a nucleotide sequence which hybridizes under stringent hybridization conditions to a complement of a nucleic acid molecule comprising the nucleotide sequence of SEQ ID NO:1 or 3.

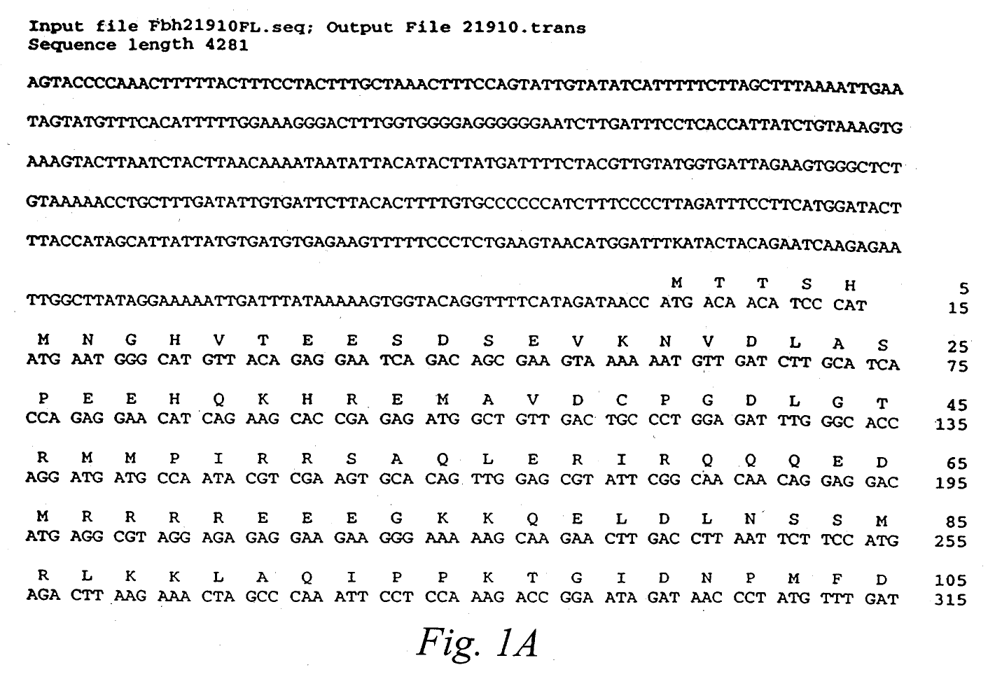

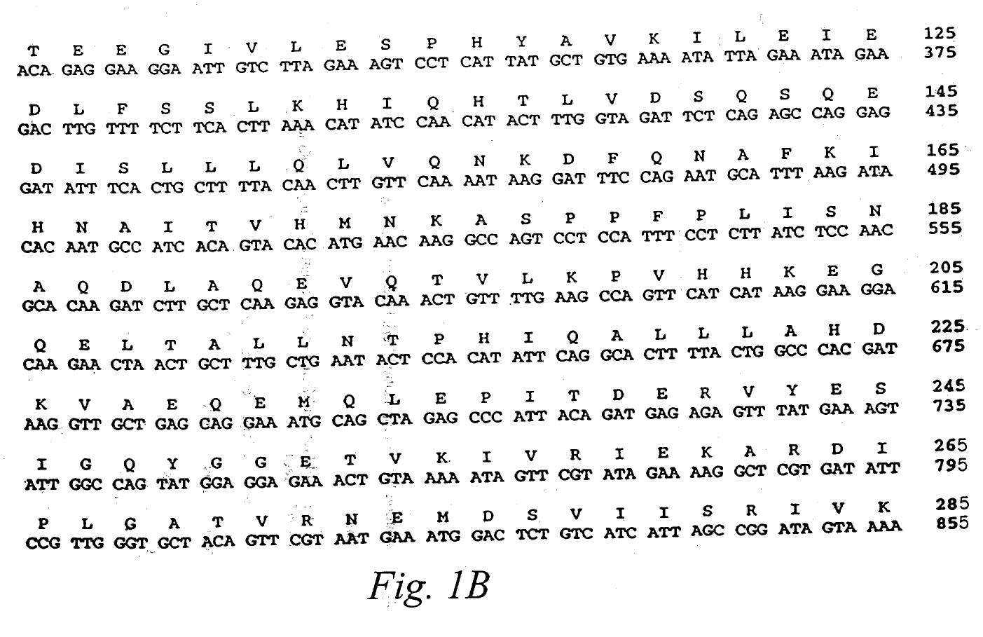

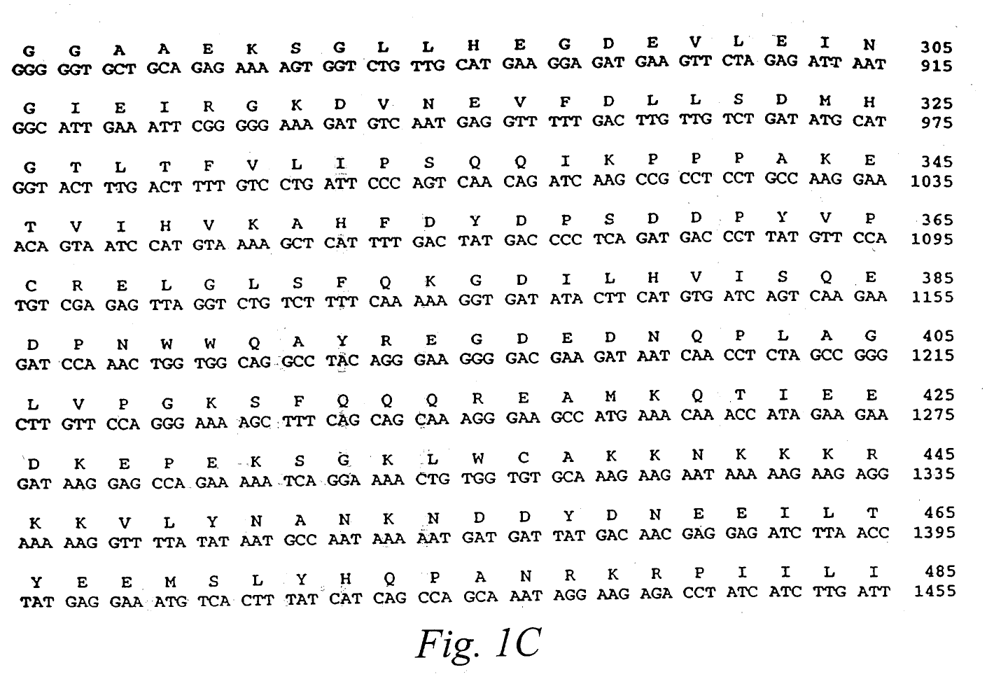

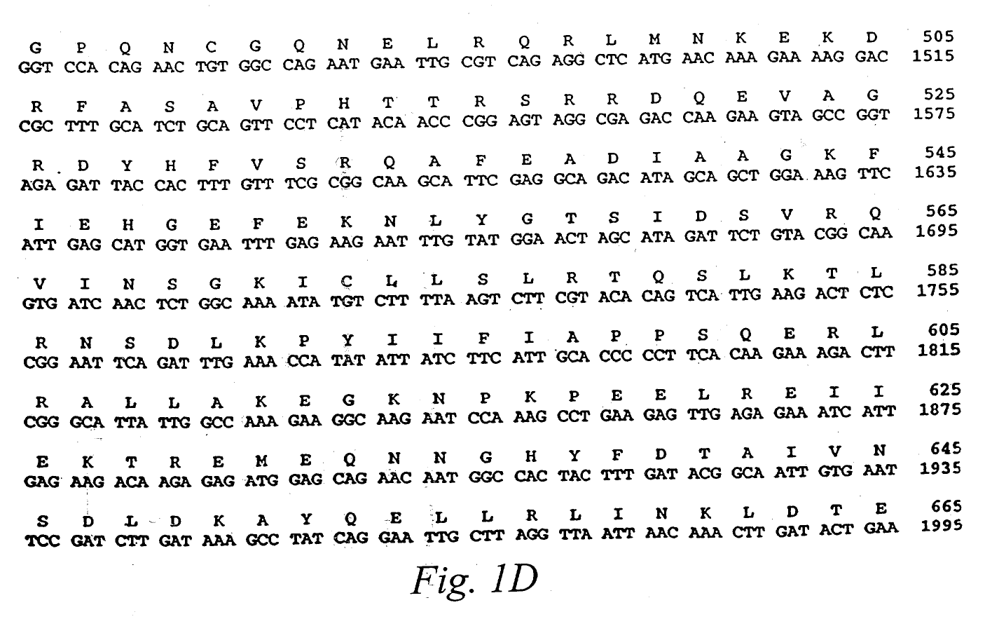

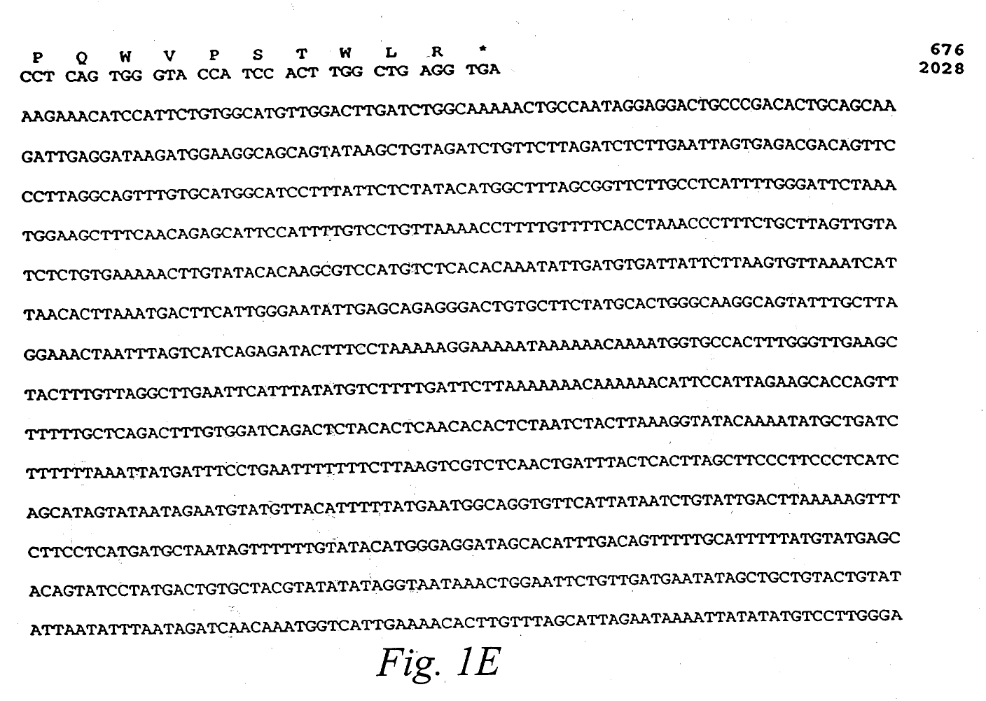

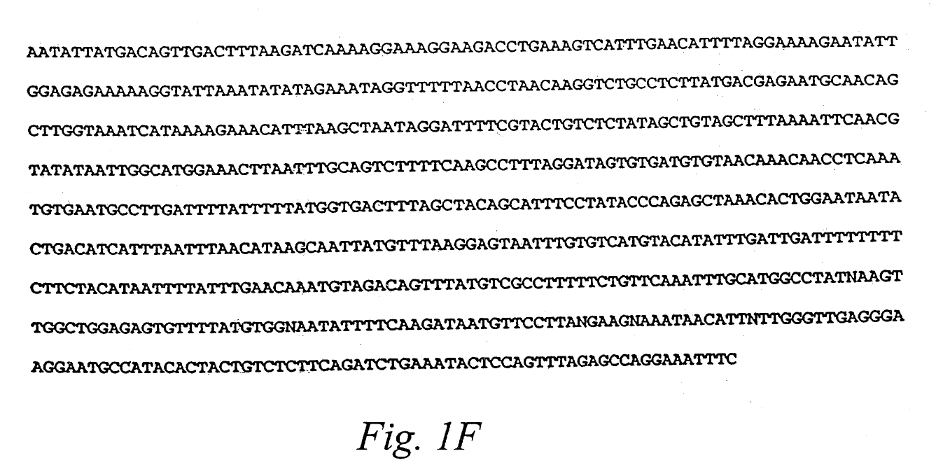

The nucleotide sequence of the isolated human MAGK cDNA and the predicted amino acid sequence of the human MAGK polypeptide are shown in FIG. 1 and in SEQ ID NOs:1 and 2, respectively. A plasmid containing the nucleotide sequence encoding human MAGK, was deposited with the American Type Culture Collection (ATCC), 10801 University Boulevard, Manassas, Va. 20110-2209, on ______ and assigned Accession Numbers ______. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The human MAGK gene, which is approximately 4281 nucleotides in length, encodes a protein having a molecular weight of approximately 74.36 kD and which is approximately 675 amino acid residues in length.

Various aspects of the invention are described in further detail in the following subsections:

Isolated Nucleic Acid Molecules:

One aspect of the invention pertains to isolated nucleic acid molecules that encode MAGK proteins or biologically active portions thereof, as well as nucleic acid fragments sufficient for use as hybridization probes to identify MAGK-encoding nucleic acid molecules (e.g., MAGK mRNA) and fragments for use as PCR primers for the amplification or mutation of MAGK nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated MAGK nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1 or 3, or the nucleotide sequence of the DNA insert of the plasmid deposited with ATCC as Accession Number ______, or a portion thereof, can be isolated using standard molecular biology techniques and the sequence information provided herein. Using all or portion of the nucleic acid sequence of SEQ ID NO:1 or 3, or the nucleotide sequence of the DNA insert of the plasmid deposited with ATCC as Accession Number ______ as a hybridization probe, MAGK nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 or 3, or the nucleotide sequence of the DNA insert of the plasmid deposited with ATCC as Accession Number ______ can be isolated by the polymerase chain reaction (PCR) using synthetic oligonucleotide primers designed based upon the sequence of SEQ ID NO:1 or 3, or the nucleotide sequence of the DNA insert of the plasmid deposited with ATCC as Accession Number ______.

A nucleic acid of the invention can be amplified using cDNA, mRNA or, alternatively, genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to MAGK nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1 or 3. This cDNA may comprise sequences encoding the human MAGK protein (i.e., “the coding region”, from nucleotides 452-2479), as well as 5′ untranslated sequences (nucleotides 1-451) and 3′ untranslated sequences (nucleotides 2480-4281) of SEQ ID NO:1. Alternatively, the nucleic acid molecule can comprise only the coding region of SEQ ID NO:1 (e.g., nucleotides 452-2479, corresponding to SEQ ID NO:3).

In another preferred embodiment, an isolated nucleic acid molecule of the invention comprises a nucleic acid molecule which is a complement of the nucleotide sequence shown in SEQ ID NO:1 or 3, or the nucleotide sequence of the DNA insert of the plasmid deposited with ATCC as Accession Number ______, or a portion of any of these nucleotide sequences. A nucleic acid molecule which is complementary to the nucleotide sequence shown in SEQ ID NO:1 or 3, or the nucleotide sequence of the DNA insert of the plasmid deposited with ATCC as Accession Number ______ , is one which is sufficiently complementary to the nucleotide sequence shown in SEQ ID NO:1 or 3, or the nucleotide sequence of the DNA insert of the plasmid deposited with ATCC as Accession Number ______ such that it can hybridize to the nucleotide sequence shown in SEQ ID NO:1 or 3, or the nucleotide sequence of the DNA insert of the plasmid deposited with ATCC as Accession Number ______, respectively, thereby forming a stable duplex.

In still another preferred embodiment, an isolated nucleic acid molecule of the present invention comprises a nucleotide sequence which is at least about 50%, 55%, 60%, 65%, 70%, 75%, 80%, 85%, 90%, 91%, 92%, 93%, 94%, 95%, 96%, 97%, 98%, 99% or more identical to the entire length of the nucleotide sequence shown in SEQ ID NO:1 or 3, or the entire length of the nucleotide sequence of the DNA insert of the plasmid deposited with ATCC as Accession Number ______, or a portion of any of these nucleotide sequences.

Moreover, the nucleic acid molecule of the invention can comprise only a portion of the nucleic acid sequence of SEQ ID NO:1 or 3, or the nucleotide sequence of the DNA insert of the plasmid deposited with ATCC as Accession Number ______, for example, a fragment which can be used as a probe or primer or a fragment encoding a portion of a MAGK protein, e.g., a biologically active portion of a MAGK protein. The nucleotide sequences determined from the cloning of the MAGK genes allow for the generation of probes and primers designed for use in identifying and/or cloning other MAGK family members, as well as MAGK homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 or 3, or the nucleotide sequence of the DNA insert of the plasmid deposited with ATCC as Accession Number ______ of an anti-sense sequence of SEQ ID NO:1 or 3, or the nucleotide sequence of the DNA insert of the plasmid deposited with ATCC as Accession Number ______ or of a naturally occurring allelic variant or mutant of SEQ ID NO:1 or 3, or the nucleotide sequence of the DNA insert of the plasmid deposited with ATCC as Accession Number _______. In one embodiment, a nucleic acid molecule of the present invention comprises a nucleotide sequence which is greater than 50-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300 or more nucleotides in length and hybridizes under stringent hybridization conditions to a nucleic acid molecule of SEQ ID NO:1 or 3, or the nucleotide sequence of the DNA insert of the plasmid deposited with ATCC as Accession Number ______.

Probes based on the MAGK nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a MAGK protein, such as by measuring a level of a MAGK-encoding nucleic acid in a sample of cells from a subject e.g., detecting MAGK mRNA levels or determining whether a genomic MAGK gene has been mutated or deleted. In one embodiment, probes or primers based on the MAGK nucleotide sequences can be used to detect MAGK transcripts in epithelial cells or tissues (e.g., lung, colon, or prostate epithelial cells or tissues, or for example, brain tissue or heart tissue.

A nucleic acid fragment encoding a “biologically active portion of a MAGK protein” can be prepared by isolating a portion of the nucleotide sequence of SEQ ID NO:1 or 3, or the nucleotide sequence of the DNA insert of the plasmid deposited with ATCC as Accession Number ______ which encodes a polypeptide having a MAGK biological activity (the biological activities of the MAGK proteins are described herein), expressing the encoded portion of the MAGK protein (e.g., by recombinant expression in vitro) and assessing the activity of the encoded portion of the MAGK protein.

The invention further encompasses nucleic acid molecules that differ from the nucleotide sequence shown in SEQ ID NO:1 or 3, or the nucleotide sequence of the DNA insert of the plasmid deposited with ATCC as Accession Number ______ due to degeneracy of the genetic code and thus encode the same MAGK proteins as those encoded by the nucleotide sequence shown in SEQ ID NO:1 or 3, or the nucleotide sequence of the DNA insert of the plasmid deposited with ATCC as Accession Number ______. In another embodiment, an isolated nucleic acid molecule of the invention has a nucleotide sequence encoding a protein having an amino acid sequence shown in SEQ ID NO:2.

In addition to the MAGK nucleotide sequences shown in SEQ ID NO:1 or 3, or the nucleotide sequence of the DNA insert of the plasmid deposited with ATCC as Accession Number ______, it will be appreciated by those skilled in the art that DNA sequence polymorphisms that lead to changes in the amino acid sequences of the MAGK proteins may exist within a population (e.g., the human population). Such genetic polymorphism in the MAGK genes may exist among individuals within a population due to natural allelic variation. As used herein, the terms “gene” and “recombinant gene” refer to nucleic acid molecules which include an open reading frame encoding a MAGK protein, preferably a mammalian MAGK protein, and can further include non-coding regulatory sequences, and introns.

Allelic variants of human MAGK include both functional and non-functional MAGK proteins. Functional allelic variants are naturally occurring amino acid sequence variants of the human MAGK protein that maintain the ability to bind a MAGK ligand or substrate and/or modulate cell proliferation and/or growth, and/or migration mechanisms. Functional allelic variants will typically contain only conservative substitution of one or more amino acids of SEQ ID NO:2, or substitution, deletion or insertion of non-critical residues in non-critical regions of the protein.

Non-functional allelic variants are naturally occurring amino acid sequence variants of the human MAGK protein that do not have the ability to either bind a MAGK ligand and/or modulate any of the MAGK activities described herein. Non-functional allelic variants will typically contain a non-conservative substitution, a deletion, or insertion or premature truncation of the amino acid sequence of SEQ ID NO:2, or a substitution, insertion or deletion in critical residues or critical regions of the protein.

The present invention further provides non-human orthologues of the human MAGK protein. Orthologues of the human MAGK protein are proteins that are isolated from non-human human organisms and possess the same MAGK ligand binding and/or modulation of membrane excitability activities of the human MAGK protein. Orthologues of the human MAGK protein can readily be identified as comprising an amino acid sequence that is substantially identical to SEQ ID NO:2.

Moreover, nucleic acid molecules encoding other MAGK family members and, thus, which have a nucleotide sequence which differs from the MAGK sequences of SEQ ID NO:1 or 3, or the nucleotide sequence of the DNA insert of the plasmid deposited with ATCC as Accession Number ______ are intended to be within the scope of the invention. For example, another MAGK cDNA can be identified based on the nucleotide sequence of human MAGK. Moreover, nucleic acid molecules encoding MAGK proteins from different species, and which, thus, have a nucleotide sequence which differs from the MAGK sequences of SEQ ID NO:1 or 3, or the nucleotide sequence of the DNA insert of the plasmid deposited with ATCC as Accession Number ______ are intended to be within the scope of the invention.

Nucleic acid molecules corresponding to natural allelic variants and homologues of the MAGK cDNAs of the invention can be isolated based on their homology to the MAGK nucleic acids disclosed herein using the cDNAs disclosed herein, or a portion thereof, as a hybridization probe according to standard hybridization techniques under stringent hybridization conditions. Nucleic acid molecules corresponding to natural allelic variants and homologues of the MAGK cDNAs of the invention can further be isolated by mapping to the same chromosome or locus as the MAGK gene.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1 or 3, or the nucleotide sequence of the DNA insert of the plasmid deposited with ATCC as Accession Number ______. In other embodiment, the nucleic acid is at least 50-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300 or more nucleotides in length.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N, where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

Preferably, an isolated nucleic acid molecule of the invention that hybridizes under stringent conditions to the sequence of SEQ ID NO:1 or 3 and corresponds to a naturally-occurring nucleic acid molecule. As used herein, a “naturally-occurring” nucleic acid molecule refers to an RNA or DNA molecule having a nucleotide

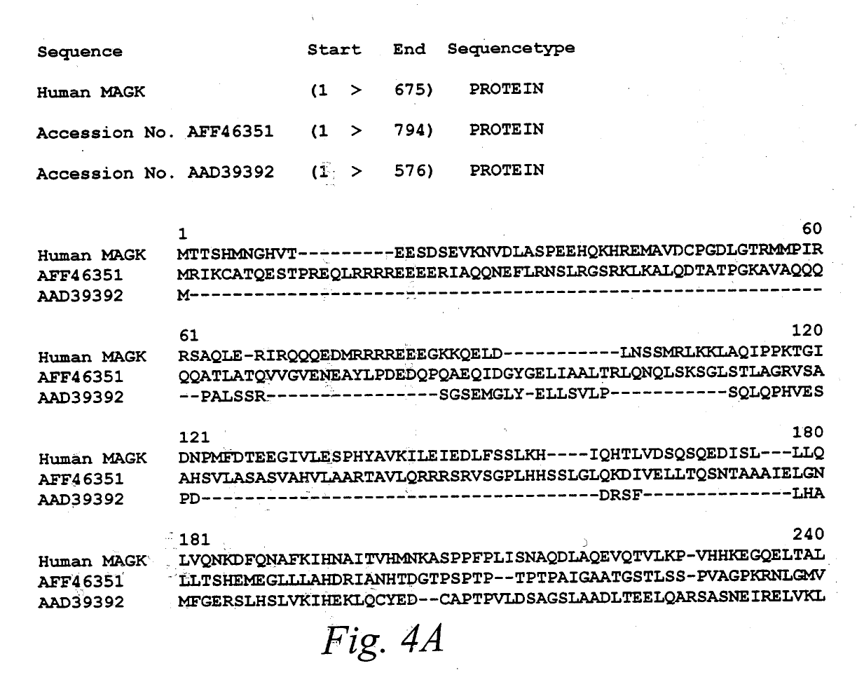

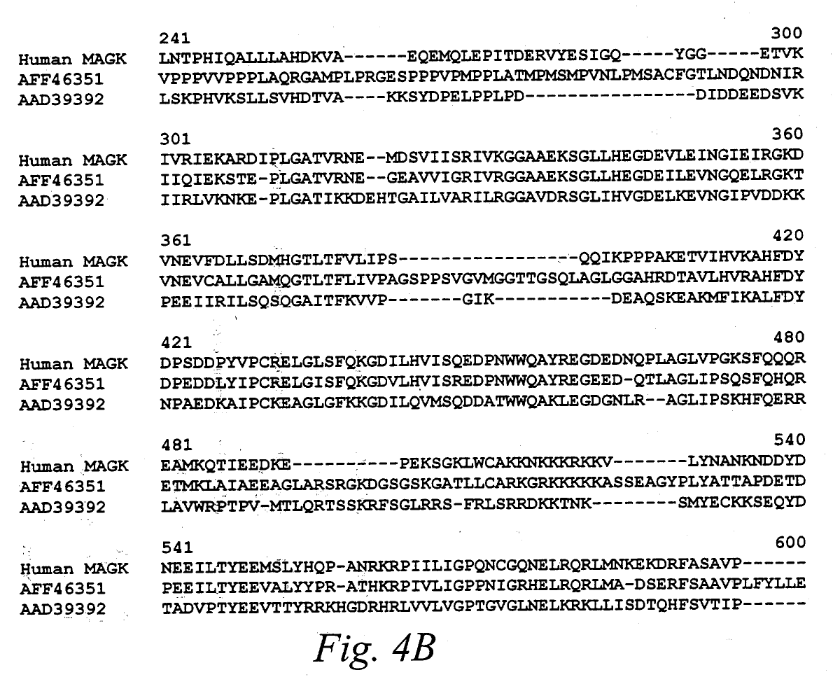

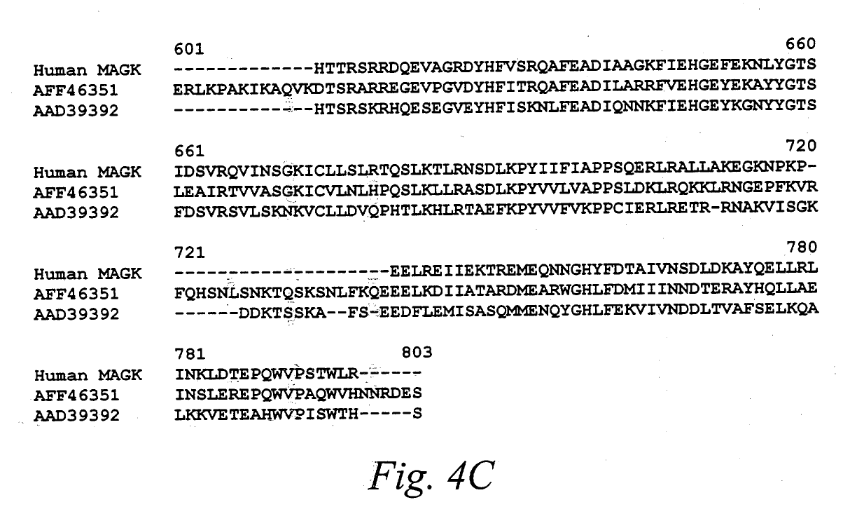

In addition to naturally-occurring allelic variants of the MAGK sequences that may exist in the population, the skilled artisan will further appreciate that changes can be introduced by mutation into the nucleotide sequences of SEQ ID NO:1 or 3, or the nucleotide sequence of the DNA insert of the plasmid deposited with ATCC as Accession Number ______, thereby leading to changes in the amino acid sequence of the encoded MAGK proteins, without altering the functional ability of the MAGK proteins. For example, nucleotide substitutions leading to amino acid substitutions at “non-essential” amino acid residues can be made in the sequence of SEQ ID NO:1 or 3, or the nucleotide sequence of the DNA insert of the plasmid deposited with ATCC as Accession Number ______. A “non-essential” amino acid residue is a residue that can be altered from the wild-type sequence of MAGK (e.g., the sequence of SEQ ID NO:2) without altering the biological activity, whereas an “essential” amino acid residue is required for biological activity. For example, amino acid residues that are conserved among the MAGK proteins of the present invention, e.g., those present in a guanylate kinase domain, are predicted to be particularly unamenable to alteration. Furthermore, additional amino acid residues that are conserved between the MAGK proteins of the present invention and other members of the MAGK family are not likely to be amenable to alteration, for example, residues conserved among the proteins aligned in FIG. 4.

Accordingly, another aspect of the invention pertains to nucleic acid molecules encoding MAGK proteins that contain changes in amino acid residues that are not essential for activity. Such MAGK proteins differ in amino acid sequence from SEQ ID NO:2, yet retain biological activity. In one embodiment, the isolated nucleic acid molecule comprises a nucleotide sequence encoding a protein, wherein the protein comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to SEQ ID NO:2.

An isolated nucleic acid molecule encoding a MAGK protein identical to the protein of SEQ ID NO:2 can be created by introducing one or more nucleotide substitutions, additions or deletions into the nucleotide sequence of SEQ ID NO:1 or 3, or the nucleotide sequence of the DNA insert of the plasmid deposited with ATCC as Accession Number ______ such that one or more amino acid substitutions, additions or deletions are introduced into the encoded protein. Mutations can be introduced into SEQ ID NO:1 or 3, or the nucleotide sequence of the DNA insert of the plasmid deposited with ATCC as Accession Number______ by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a MAGK protein is preferably replaced with another amino acid residue from the same side chain family. Alternatively, in another embodiment, mutations can be introduced randomly along all or part of a MAGK coding sequence, such as by saturation mutagenesis, and the resultant mutants can be screened for MAGK biological activity to identify mutants that retain activity. Following mutagenesis of SEQ ID NO:1 or 3, or the nucleotide sequence of the DNA insert of the plasmid deposited with ATCC as Accession Number ______ , the encoded protein can be expressed recombinantly and the activity of the protein can be determined.

In a preferred embodiment, a mutant MAGK protein can be assayed for the ability to metabolize or catabolize biochemical molecules necessary for energy production or storage, permit intra- or intercellular signaling, e.g., cell growth signaling, metabolize or catabolize metabolically important biomolecules, and to detoxify, potentially harmful compounds.

In addition to the nucleic acid molecules encoding MAGK proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire MAGK coding strand, or to only a portion thereof. In one embodiment, an antisense nucleic acid molecule is antisense to a “coding region” of the coding strand of a nucleotide sequence encoding a MAGK. The term “coding region” refers to the region of the nucleotide sequence comprising codons which are translated into amino acid residues (e.g., the coding region of human MAGK corresponds to SEQ ID NO:3). In another embodiment, the antisense nucleic acid molecule is antisense to a “noncoding region” of the coding strand of a nucleotide sequence encoding MAGK. The term “noncoding region” refers to 5′ and 3′ sequences which flank the coding region that are not translated into amino acids (i.e., also referred to as 5′ and 3′ untranslated regions).

Given the coding strand sequences encoding MAGK disclosed herein (e.g., SEQ ID NO:3), antisense nucleic acids of the invention can be designed according to the rules of Watson and Crick base pairing. The antisense nucleic acid molecule can be complementary to the entire coding region of MAGK mRNA, but more preferably is an oligonucleotide which is antisense to only a portion of the coding or noncoding region of MAGK mRNA. For example, the antisense oligonucleotide can be complementary to the region surrounding the translation start site of MAGK mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i. e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a MAGK protein to thereby inhibit expression of the protein, e.g., by inhibiting transcription and/or translation, thereby inhibiting MAGK associated activity. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591) or hairpin ribozymes (described in Fedor (2000) J Mol Biol 297(2):269)) can be used to catalytically cleave MAGK mRNA transcripts to thereby inhibit translation of MAGK mRNA. A ribozyme having specificity for a MAGK-encoding nucleic acid can be designed based upon the nucleotide sequence of a MAGK cDNA disclosed herein (i. e., SEQ ID NO:1 or 3, or the nucleotide sequence of the DNA insert of the plasmid deposited with ATCC as Accession Number ______ ). For example, a derivative of a Tetrahymena L-19 IVS RNA can be constructed in which the nucleotide sequence of the active site is complementary to the nucleotide sequence to be cleaved in a MAGK-encoding mRNA. See, e.g., Cech et al. U.S. Pat. No. 4,987,071; and Cech et al. U.S. Pat. No. 5,116,742. Alternatively, MAGK mRNA can be used to select a catalytic RNA having a specific ribonuclease activity from a pool of RNA molecules. See, e.g., Bartel, D. and Szostak, J. W. (1993) Science 261:1411-1418.

Alternatively, MAGK gene expression can be inhibited by targeting nucleotide sequences complementary to the regulatory region of the MAGK (e.g., the MAGK promoter and/or enhancers; e.g., nucleotides 1-451 of SEQ ID NO:1) to form triple helical structures that prevent transcription of the MAGK gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioassays 14(12):807-15.

In yet another embodiment, the MAGK nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of MAGK nucleic acid molecules can be used in therapeutic and diagnostic applications. For example, PNAs can be used as antisense or antigene agents for sequence-specific specific modulation of gene expression by, for example, inducing transcription or translation arrest or inhibiting replication. PNAs of MAGK nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of MAGK can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of MAGK nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

Alternatively, the expression characteristics of an endogenous MAGK gene within a cell line or microorganism may be modified by inserting a heterologous DNA regulatory element into the genome of a stable cell line or cloned microorganism such that the inserted regulatory element is operatively linked with the endogenous MAGK gene. For example, an endogenous MAGK gene which is normally “transcriptionally silent”, i.e., a MAGK gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous MAGK gene may be activated by insertion of a promiscuous regulatory element that works across cell types.

A heterologous regulatory element may be inserted into a stable cell line or cloned microorganism, such that it is operatively linked with an endogenous MAGK gene, using techniques, such as targeted homologous recombination, which are well known to those of skill in the art, and described, e.g., in Chappel, U.S. Pat. No. 5,272,071; PCT publication No. WO 91/06667, published May 16, 1991.

II. Isolated MAGK Proteins and Anti-MAGK Antibodies

One aspect of the invention pertains to isolated MAGK proteins, and biologically active portions thereof, as well as polypeptide fragments suitable for use as immunogens to raise anti-MAGK antibodies. In one embodiment, native MAGK proteins can be isolated from cells or tissue sources by an appropriate purification scheme using standard protein purification techniques. In another embodiment, MAGK proteins are produced by recombinant DNA techniques. Alternative to recombinant expression, a MAGK protein or polypeptide can be synthesized chemically using standard peptide synthesis techniques.

An “isolated” or “purified” protein or biologically active portion thereof is substantially free of cellular material or other contaminating proteins from the cell or tissue source from which the MAGK protein is derived, or substantially free from chemical precursors or other chemicals when chemically synthesized. The language “substantially free of cellular material” includes preparations of MAGK protein in which the protein is separated from cellular components of the cells from which it is isolated or recombinantly produced. In one embodiment, the language “substantially free of cellular material” includes preparations of MAGK protein having less than about 30% (by dry weight) of non-MAGK protein (also referred to herein as a “contaminating protein”), more preferably less than about 20% of non-MAGK protein, still more preferably less than about 10% of non-MAGK protein, and most preferably less than about 5% non-MAGK protein. When the MAGK protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of MAGK protein in which the protein is separated from chemical precursors or other chemicals which are involved in the synthesis of the protein. In one embodiment, the language “substantially free of chemical precursors or other chemicals” includes preparations of MAGK protein having less than about 30% (by dry weight) of chemical precursors or non-MAGK chemicals, more preferably less than about 20% chemical precursors or non-MAGK chemicals, still more preferably less than about 10% chemical precursors or non-MAGK chemicals, and most preferably less than about 5% chemical precursors or non-MAGK chemicals.

As used herein, a “biologically active portion” of a MAGK protein includes a fragment of a MAGK protein which participates in an interaction between a MAGK molecule and a non-MAGK molecule. Biologically active portions of a MAGK protein include peptides comprising amino acid sequences sufficiently identical to or derived from the amino acid sequence of the MAGK protein, e.g., the amino acid sequence shown in SEQ ID NO:2, which include less amino acids than the full length MAGK proteins, and exhibit at least one activity of a MAGK protein. Typically, biologically active portions comprise a domain or motif with at least one activity of the MAGK protein, e.g., modulating membrane excitability or modulating cell signaling. A biologically active portion of a MAGK protein can be a polypeptide which is, for example, 25, 50, 75, 100, 125, 150, 175, 200, 250, 300 or more amino acids in length. Biologically active portions of a MAGK protein can be used as targets for developing agents which modulate a MAGK mediated activity, e.g., cellular growth or proliferation.

In one embodiment, a biologically active portion of a MAGK protein comprises at least one guanylate kinase domain. It is to be understood that a preferred biologically active portion of a MAGK protein of the present invention may contain at least one or more of the following domains: an ATP/GTP-binding site motif A (P-loop), a guanylate kinase domain, a PDZ domain, and a SH3 domain. Moreover, other biologically active portions, in which other regions of the protein are deleted, can be prepared by recombinant techniques and evaluated for one or more of the functional activities of a native MAGK protein.

In a preferred embodiment, the MAGK protein has an amino acid sequence shown in SEQ ID NO:2. In other embodiments, the MAGK protein is substantially identical to SEQ ID NO:2, and retains the functional activity of the protein of SEQ ID NO:2, yet differs in amino acid sequence due to natural allelic variation or mutagenesis, as described in detail in subsection I above. Accordingly, in another embodiment, the MAGK protein is a protein which comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the MAGK amino acid sequence of SEQ ID NO:2 having 676 amino acid residues, at least 100, preferably at least 150, more preferably at least 200, even more preferably at least 250, and even more preferably at least 300 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to MAGK nucleic acid molecules of the invention. BLAST protein searches can be performed with the XBLAST program, score=100, wordlength=3 to obtain amino acid sequences homologous to MAGK protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also provides MAGK chimeric or fusion proteins. As used herein, a MAGK “chimeric protein” or “fusion protein” comprises a MAGK polypeptide operatively linked to a non-MAGK polypeptide. A “MAGK polypeptide” refers to a polypeptide having an amino acid sequence corresponding to a MAGK molecule, whereas a “non-MAGK polypeptide” refers to a polypeptide having an amino acid sequence corresponding to a protein which is not substantially homologous to the MAGK protein, e.g., a protein which is different from the MAGK protein and which is derived from the same or a different organism. Within a MAGK fusion protein the MAGK polypeptide can correspond to all or a portion of a MAGK protein. In a preferred embodiment, a MAGK fusion protein comprises at least one biologically active portion of a MAGK protein. In another preferred embodiment, a MAGK fusion protein comprises at least two biologically active portions of a MAGK protein. Within the fusion protein, the term “operatively linked” is intended to indicate that the MAGK polypeptide and the non-MAGK polypeptide are fused in-frame to each other. The non-MAGK polypeptide can be fused to the N-terminus or C-terminus of the MAGK polypeptide.

For example, in one embodiment, the fusion protein is a GST-MAGK fusion protein in which the MAGK sequences are fused to the C-terminus of the GST sequences. Such fusion proteins can facilitate the purification of recombinant MAGK.

In another embodiment, the fusion protein is a MAGK protein containing a heterologous signal sequence at its N-terminus. In certain host cells (e.g., mammalian host cells), expression and/or secretion of MAGK can be increased through use of a heterologous signal sequence.

The MAGK fusion proteins of the invention can be incorporated into pharmaceutical compositions and administered to a subject in vivo. The MAGK fusion proteins can be used to affect the bioavailability of a MAGK substrate. Use of MAGK fusion proteins may be useful therapeutically for the treatment of disorders caused by, for example, (i) aberrant modification or mutation of a gene encoding a MAGK protein; (ii) mis-regulation of the MAGK gene; and (iii) aberrant post-translational modification of a MAGK protein.

Moreover, the MAGK-fusion proteins of the invention can be used as immunogens to produce anti-MAGK antibodies in a subject, to purify MAGK ligands and in screening assays to identify molecules which inhibit the interaction of MAGK with a MAGK substrate.

Preferably, a MAGK chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A MAGK-encoding nucleic acid can be cloned into such an expression vector such that the fusion moiety is linked in-frame to the MAGK protein.

The present invention also pertains to variants of the MAGK proteins which function as either MAGK agonists (mimetics) or as MAGK antagonists. Variants of the MAGK proteins can be generated by mutagenesis, e.g., discrete point mutation or truncation of a MAGK protein. An agonist of the MAGK proteins can retain substantially the same, or a subset, of the biological activities of the naturally occurring form of a MAGK protein. An antagonist of a MAGK protein can inhibit one or more of the activities of the naturally occurring form of the MAGK protein by, for example, competitively modulating a MAGK-mediated activity of a MAGK protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the MAGK protein.

In one embodiment, variants of a MAGK protein which function as either MAGK agonists (mimetics) or as MAGK antagonists can be identified by screening combinatorial libraries of mutants, e.g., truncation mutants, of a MAGK protein for MAGK protein agonist or antagonist activity. In one embodiment, a variegated library of MAGK variants is generated by combinatorial mutagenesis at the nucleic acid level and is encoded by a variegated gene library. A variegated library of MAGK variants can be produced by, for example, enzymatically ligating a mixture of synthetic oligonucleotides into gene sequences such that a degenerate set of potential MAGK sequences is expressible as individual polypeptides, or alternatively, as a set of larger fusion proteins (e.g., for phage display) containing the set of MAGK sequences therein. There are a variety of methods which can be used to produce libraries of potential MAGK variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MAGK sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a MAGK protein coding sequence can be used to generate a variegated population of MAGK fragments for screening and subsequent selection of variants of a MAGK protein. In one embodiment, a library of coding sequence fragments can be generated by treating a double stranded PCR fragment of a MAGK coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the MAGK protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of MAGK proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify MAGK variants (Arkin and Yourvan (1992) Proc. Natl. Acad. Sci. USA 89:7811-7815; Delgrave et al. (1993) Protein Engineering 6(3): 327-331).

In one embodiment, cell based assays can be exploited to analyze a variegated MAGK library. For example, a library of expression vectors can be transfected into a cell line, e.g., a neuronal cell line, which ordinarily responds to a MAGK ligand in a particular MAGK ligand-dependent manner. The transfected cells are then contacted with a MAGK ligand and the effect of expression of the mutant on, eg., membrane excitability of MAGK can be detected. Plasmid DNA can then be recovered from the cells which score for inhibition, or alternatively, potentiation of signaling by the MAGK ligand, and the individual clones further characterized.

An isolated MAGK protein, or a portion or fragment thereof, can be used as an immunogen to generate antibodies that bind MAGK using standard techniques for polyclonal and monoclonal antibody preparation. A full-length MAGK protein can be used or, alternatively, the invention provides antigenic peptide fragments of MAGK for use as immunogens. The antigenic peptide of MAGK comprises at least 8 amino acid residues of the amino acid sequence shown in SEQ ID NO:2 and encompasses an epitope of MAGK such that an antibody raised against the peptide forms a specific immune complex with the MAGK protein. Preferably, the antigenic peptide comprises at least 10 amino acid residues, more preferably at least 15 amino acid residues, even more preferably at least 20 amino acid residues, and most preferably at least 30 amino acid residues.

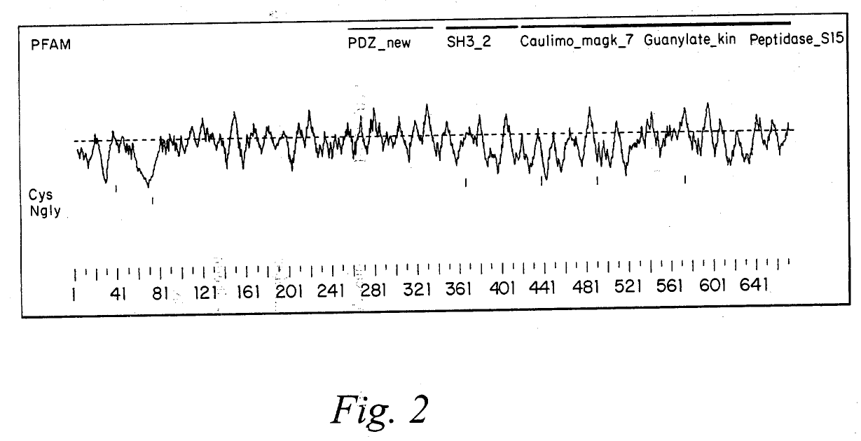

Preferred epitopes encompassed by the antigenic peptide are regions of MAGK that are located on the surface of the protein, e.g., hydrophilic regions, as well as regions with high antigenicity (see, for example, FIG. 2).

A MAGK immunogen typically is used to prepare antibodies by immunizing a suitable subject, (e.g., rabbit, goat, mouse or other mammal) with the immunogen. An appropriate immunogenic preparation can contain, for example, recombinantly expressed MAGK protein or a chemically synthesized MAGK polypeptide. The preparation can further include an adjuvant, such as Freund's complete or incomplete adjuvant, or similar immunostimulatory agent. Immunization of a suitable subject with an immunogenic MAGK preparation induces a polyclonal anti-MAGK antibody response.

Accordingly, another aspect of the invention pertains to anti-MAGK antibodies. The term “antibody” as used herein refers to immunoglobulin molecules and immunologically active portions of immunoglobulin molecules, i.e., molecules that contain an antigen binding site which specifically binds (immunoreacts with) an antigen, such as a MAGK. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind MAGK molecules. The term “monoclonal antibody” or “monoclonal antibody composition”, as used herein, refers to a population of antibody molecules that contain only one species of an antigen binding site capable of immunoreacting with a particular epitope of MAGK. A monoclonal antibody composition thus typically displays a single binding affinity for a particular MAGK protein with which it immunoreacts.

Polyclonal anti-MAGK antibodies can be prepared as described above by immunizing a suitable subject with a MAGK immunogen. The anti-MAGK antibody titer in the immunized subject can be monitored over time by standard techniques, such as with an enzyme linked immunosorbent assay (ELISA) using immobilized MAGK. If desired, the antibody molecules directed against MAGK can be isolated from the mammal (e.g., from the blood) and further purified by well known techniques, such as protein A chromatography to obtain the IgG fraction. At an appropriate time after immunization, e.g., when the anti-MAGK antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem.255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a MAGK immunogen as described above, and the culture supernatants of the resulting hybridoma cells are screened to identify a hybridoma producing a monoclonal antibody that binds MAGK.

Any of the many well known protocols used for fusing lymphocytes and immortalized cell lines can be applied for the purpose of generating an anti-MAGK monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind MAGK, e.g., using a standard ELISA assay.

Alternative to preparing monoclonal antibody-secreting hybridomas, a monoclonal anti-MAGK antibody can be identified and isolated by screening a recombinant combinatorial immunoglobulin library (e.g., an antibody phage display library) with MAGK to thereby isolate immunoglobulin library members that bind MAGK. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-MAGK antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. 1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-MAGK antibody (e.g., monoclonal antibody) can be used to isolate MAGK by standard techniques, such as affinity chromatography or immunoprecipitation. An anti-MAGK antibody can facilitate the purification of natural MAGK from cells and of recombinantly produced MAGK expressed in host cells. Moreover, an anti-MAGK antibody can be used to detect MAGK protein (e.g., in a cellular lysate or cell supernatant) in order to evaluate the abundance and pattern of expression of the MAGK protein. Anti-MAGK antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a MAGK protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., MAGK proteins, mutant forms of MAGK proteins, fusion proteins, and the like).

The recombinant expression vectors of the invention can be designed for expression of MAGK proteins in prokaryotic or eukaryotic cells. For example, MAGK proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in MAGK activity assays, (e.g., direct assays or competitive assays described in detail below), or to generate antibodies specific for MAGK proteins, for example. In a preferred embodiment, a MAGK fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the MAGK expression vector is a yeast expression vector. Examples of vectors for expression in yeast S. cerevisiae include pYepSec 1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, MAGK proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the (α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to MAGK mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a MAGK nucleic acid molecule of the invention is introduced, e.g., a MAGK nucleic acid molecule within a recombinant expression vector or a MAGK nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a MAGK protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a MAGK protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a MAGK protein. Accordingly, the invention further provides methods for producing a MAGK protein using the host cells of the invention. In one embodiment, the method comprises culturing the host cell of the invention (into which a recombinant expression vector encoding a MAGK protein has been introduced) in a suitable medium such that a MAGK protein is produced. In another embodiment, the method further comprises isolating a MAGK protein from the medium or the host cell.

The host cells of the invention can also be used to produce non-human transgenic animals. For example, in one embodiment, a host cell of the invention is a fertilized oocyte or an embryonic stem cell into which MAGK-coding sequences have been introduced. Such host cells can then be used to create non-human transgenic animals in which exogenous MAGK sequences have been introduced into their genome or homologous recombinant animals in which endogenous MAGK sequences have been altered. Such animals are useful for studying the function and/or activity of a MAGK and for identifying and/or evaluating modulators of MAGK activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous MAGK gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a MAGK-encoding nucleic acid into the male pronuclei of a fertilized oocyte, e.g., by microinjection, retroviral infection, and allowing the oocyte to develop in a pseudopregnant female foster animal. The MAGK cDNA sequence of SEQ ID NO:1 can be introduced as a transgene into the genome of a non-human animal. Alternatively, a nonhuman homologue of a human MAGK gene, such as a mouse or rat MAGK gene, can be used as a transgene. Alternatively, a MAGK gene homologue, such as another MAGK family member can be isolated based on hybridization to the MAGK cDNA sequences of SEQ ID NO:1 or 3, or the DNA insert of the plasmid deposited with ATCC as Accession Number ______ (described further in subsection I above) and used as a transgene. Intronic sequences and polyadenylation signals can also be included in the transgene to increase the efficiency of expression of the transgene. A tissue-specific regulatory sequence(s) can be operably linked to a MAGK transgene to direct expression of a MAGK protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a MAGK transgene in its genome and/or expression of MAGK mRNA in tissues or cells of the animals. A transgenic founder animal can then be used to breed additional animals carrying the transgene. Moreover, transgenic animals carrying a transgene encoding a MAGK protein can further be bred to other transgenic animals carrying other transgenes.

To create a homologous recombinant animal, a vector is prepared which contains at least a portion of a MAGK gene into which a deletion, addition or substitution has been introduced to thereby alter, e.g., functionally disrupt, the MAGK gene. The MAGK gene can be a human gene (e.g., the cDNA of SEQ ID NO:3), but more preferably, is a non-human homologue of a human MAGK gene (e.g., a cDNA isolated by stringent hybridization with the nucleotide sequence of SEQ ID NO:1). For example, a mouse MAGK gene can be used to construct a homologous recombination nucleic acid molecule, e.g., a vector, suitable for altering an endogenous MAGK gene in the mouse genome. In a preferred embodiment, the homologous recombination nucleic acid molecule is designed such that, upon homologous recombination, the endogenous MAGK gene is functionally disrupted (i.e., no longer encodes a functional protein; also referred to as a “knock out” vector). Alternatively, the homologous recombination nucleic acid molecule can be designed such that, upon homologous recombination, the endogenous MAGK gene is mutated or otherwise altered but still encodes functional protein (e.g., the upstream regulatory region can be altered to thereby alter the expression of the endogenous MAGK protein). In the homologous recombination nucleic acid molecule, the altered portion of the MAGK gene is flanked at its 5′ and 3′ ends by additional nucleic acid sequence of the MAGK gene to allow for homologous recombination to occur between the exogenous MAGK gene carried by the homologous recombination nucleic acid molecule and an endogenous MAGK gene in a cell, e.g., an embryonic stem cell. The additional flanking MAGK nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) i Cell 51:503 for a description of homologous recombination vectors). The homologous recombination nucleic acid molecule is introduced into a cell, e.g., an embryonic stem cell line (e.g., by electroporation) and cells in which the introduced MAGK gene has homologously recombined with the endogenous MAGK gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells:A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The MAGK nucleic acid molecules, MAGK antisense molecules, ribozymes, and or MAGK modulators (e.g., small molecule modulators), fragments of MAGK proteins, and anti-MAGK antibodies (also referred to herein as “active compounds”) of the invention can be incorporated into pharmaceutical compositions suitable for administration. Such compositions typically comprise the nucleic acid molecule, protein, antibody, ribozyme or modulator, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a MAGK nucleic acid molecule, antisense molecule, a fragment of a MAGK protein or an anti-MAGK antibody, ribozyme or MAGK modulator) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In certain embodiments of the invention, a modulator of MAGK activity is administered in combination with other agents (e.g., a small molecule), or in conjunction with another, complementary treatment regime. For example, in one embodiment, a modulator of MAGK activity is used to treat a MAGK associated disorder. Accordingly, modulation of MAGK activity may be used in conjunction with, for example, another agent used to treat the MAGK associated disorder, e.g., another known agent used to treat cancer, in particular, lung cancer or colon cancer.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, antibodies, ribozymes, and/or modulators described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a MAGK protein of the invention has one or more of the following activities: 1) modulate ATP-dependent phosphorylation of GMP or dGMP, 2) modulate intra-or intercellular signaling, e.g., cellular growth signaling, 3) modulate metabolism or catabolism of metabolically important biomolecules (e.g., nucleotides), 4) modulate cellular growth and differentiation, and 5) modulate cellular proliferation and 6) modulate signal transduction.

Accordingly, the MAGK nucleic acid molecules, antisense molecules, proteins, protein fragments, ribozymes, antibodies, and/or MAGK modulators (e.g., small molecule modulators) can be used to regulate and/or modulate any of the MAGK activities described herein and/or to modulate or regulate, diagnose or treat any disorder associated with a MAGK activity, as described herein.

As used herein, a “membrane-associated guanylate kinase-associated disorder” includes a disorder, disease or condition which is caused or characterized by a misregulation (e.g., downregulation or upregulation) of membrane-associated guanylate kinase activity. Misregulation of membrane-associated guanylate kinase activity can result in the overproduction or lack of production of nucleotides and metabolic energy for the cell, or in aberrant cellular signaling, e.g., cellular growth signaling. Membrane-associated guanylate kinase-associated disorders, therefore, can detrimentally affect cellular functions such as cellular proliferation, growth, differentiation, or migration, inter- or intra-cellular communication; and tissue function. Examples of membrane-associated guanylate kinase-associated disorders include, but are not limited to, cellular growth or proliferation diseases or disorders. Cellular growth or proliferation diseases or disorders include those disorders that affect cell growth or proliferation processes. Such diseases and disorders include, but are not limited to, cancer, e.g., carcinoma, sarcoma, or leukemia, examples of which include, but are not limited to, colon, lung, liver, ovary, and breast; tumorigenesis and metastasis; skeletal dysplasia; hepatic disorders; and hematopoietic and/or myeloproliferative disorders. MAGK-associated or related disorders also include disorders affecting tissues in which MAGK protein is expressed (see Example 1).

The isolated nucleic acid molecules of the invention can be used, for example, to express MAGK protein (e.g., via a recombinant expression vector in a host cell in gene therapy applications), to detect MAGK mRNA (e.g., in a biological sample) or a genetic alteration in a MAGK gene, and to modulate MAGK activity, as described further below. The MAGK proteins can be used to treat disorders characterized by insufficient or excessive production of a MAGK substrate or production of MAGK inhibitors. In addition, the MAGK proteins can be used to screen for naturally occurring MAGK substrates, to screen for drugs or compounds which modulate MAGK activity, as well as to treat disorders characterized by insufficient or excessive production of MAGK protein or production of MAGK protein forms which have decreased, aberrant or unwanted activity compared to MAGK wild type protein (e.g., guanylate kinase-associated disorders, for example, guanylate kinase disorders). Moreover, the anti-MAGK antibodies of the invention can be used to detect and isolate MAGK proteins, regulate the bioavailability of MAGK proteins, and modulate MAGK activity.

In yet another embodiment, the MAGK molecules of the invention are useful for catalyzing the ATP-dependent phosphorylation of either GMP to GDP or dGMP to dGDP. As such, these molecules may be employed in small or large-scale synthesis of either GDP or dGDP, or in chemical processes that require the production or interconversion of these compounds. Such processes are known in the art (see, e.g., Ullmann et al. (1999) Ullmann's Encyclopedia of Industrial Chemistry, 6^{th} ed. VCH: Weinheim; Gutcho (1983) Chemicals by Fermentation. Park ridge, N.J.: Noyes Data Corporation (ISBN 0818805086); Rehm et al. (eds.) (1993) Biotechnology, 2^{nd} ed. VCH: Weinheim; and Michal, G. (1999) Biochemical Pathways: An Atlas of Biochemistry and Molecular Biology. New York: John Wiley & Sons, and references contained therein.)

A. Screening Assays:

The invention provides a method (also referred to herein as a “screening assay”) for identifying modulators, i e., candidate or test compounds or agents (e.g., peptides, peptidomimetics, small molecules (organic or inorganic) or other drugs) which bind to MAGK proteins, have a stimulatory or inhibitory effect on, for example, MAGK expression or MAGK activity, or have a stimulatory or inhibitory effect on, for example, the expression or activity of MAGK substrate.

These assays are designed to identify compounds that bind to a MAGK protein, bind to other inter- or extra-cellular proteins that interact with a MAGK protein, and/or interfere with the interaction of the MAGK protein with other inter- or extra-cellular proteins. For example, in the case of the MAGK protein, which is protein that is capable of interaction with the cytoskeleton of the cell to regulate cellular growth and proliferation, signaling mechanisms, e.g., cellular growth signaling mechanisms, transcription, and intercellular junctions, such techniques can be used to identify ligands for such a protein. A MAGK protein modulator can, for example, be used to ameliorate cellular growth or proliferation diseases or disorders, e.g., cancer. Such compounds may include, but are not limited to MAGK peptides, anti-MAGK antibodies, or small organic or inorganic compounds. Such compounds may also include other cellular proteins or peptides.

Compounds identified via assays such as those described herein may be useful, for example, for ameliorating cellular growth and proliferation diseases or disorders. In instances whereby a cellular growth or proliferation disease condition results from an overall lower level of MAGK gene expression and/or MAGK protein in a cell or tissue, compounds that interact with the MAGK protein may include compounds which accentuate or amplify the activity of the bound MAGK protein. Such compounds would bring about an effective increase in the level of MAGK protein activity, thus ameliorating symptoms.

In other instances, mutations within the MAGK gene may cause aberrant types or excessive amounts of MAGK proteins to be made which have a deleterious effect that leads to a cellular growth or proliferation disease or disorder. Similarly, physiological conditions may cause an excessive increase in MAGK gene expression leading to a cellular growth or proliferation disease or disorder. In such cases, compounds that bind to a MAGK protein may be identified that inhibit the activity of the MAGK protein. Assays for testing the effectiveness of compounds identified by techniques such as those described in this section are discussed herein.

In one embodiment, the invention provides assays for screening candidate or test compounds which are substrates of a MAGK protein or polypeptide or biologically active portion thereof (e.g., GMP or dGMP). In another embodiment, the invention provides assays for screening candidate or test compounds which bind to or modulate the activity of a MAGK protein or polypeptide or biologically active portion thereof (e.g., ATP, inhibitory molecules). The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a MAGK protein or biologically active portion thereof is contacted with a test compound and the ability of the test compound to modulate MAGK activity is determined. Determining the ability of the test compound to modulate MAGK activity can be accomplished by monitoring, for example, the production of one or more specific metabolites in a cell which expresses MAGK (see, e.g., Saada et al. (2000) Biochem Biophys. Res. Commun. 269: 382-386), cell growth and/or proliferation, or cell signaling. The cell, for example, can be of mammalian origin, e.g., an epithelial cell, for example a lung or colon epithelial cell, or a tumor cell. The ability of the test compound to modulate MAGK binding to a substrate (e.g., GMP or dGMP) or to bind to MAGK can also be determined. Determining the ability of the test compound to modulate MAGK binding to a substrate can be accomplished, for example, by coupling the MAGK substrate with a radioisotope or enzymatic label such that binding of the MAGK substrate to MAGK can be determined by detecting the labeled MAGK substrate in a complex. Alternatively, MAGK could be coupled with a radioisotope or enzymatic label to monitor the ability of a test compound to modulate MAGK binding to a MAGK substrate in a complex. Determining the ability of the test compound to bind MAGK can be accomplished, for example, by coupling the compound with a radioisotope or enzymatic label such that binding of the compound to MAGK can be determined by detecting the labeled MAGK compound in a complex. For example, compounds (e.g., MAGK substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a MAGK substrate) to interact with MAGK without the labeling of any of the interactants. For example, a microphysiometer can be used to detect the interaction of a compound with MAGK without the labeling of either the compound or the MAGK. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and MAGK.

In another embodiment, an assay is a cell-based assay comprising contacting a cell expressing a MAGK target molecule (e.g., a MAGK substrate) with a test compound and determining the ability of the test compound to modulate (e.g., stimulate or inhibit) the activity of the MAGK target molecule. Determining the ability of the test compound to modulate the activity of a MAGK target molecule can be accomplished, for example, by determining the ability of the MAGK protein to bind to or interact with the MAGK target molecule.

Determining the ability of the MAGK protein, or a biologically active fragment thereof, to bind to or interact with a MAGK target molecule can be accomplished by one of the methods described above for determining direct binding. In a preferred embodiment, determining the ability of the MAGK protein to bind to or interact with a MAGK target molecule can be accomplished by determining the activity of the target molecule. For example, the activity of the target molecule can be determined by detecting induction of a cellular response (i.e., changes in intracellular K⁺ levels), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., cell growth or proliferation).

In yet another embodiment, an assay of the present invention is a cell-free assay in which a MAGK protein or biologically active portion thereof is contacted with a test compound and the ability of the test compound to bind to the MAGK protein or biologically active portion thereof is determined. Preferred biologically active portions of the MAGK proteins to be used in assays of the present invention include fragments which participate in interactions with non-MAGK molecules, e.g., fragments with high surface probability scores (see, for example, FIG. 2). Binding of the test compound to the MAGK protein can be determined either directly or indirectly as described above. In a preferred embodiment, the assay includes contacting the MAGK protein or biologically active portion thereof with a known compound which binds MAGK to form an assay mixture, contacting the assay mixture with a test compound, and determining the ability of the test compound to interact with a MAGK protein, wherein determining the ability of the test compound to interact with a MAGK protein comprises determining the ability of the test compound to preferentially bind to MAGK or biologically active portion thereof as compared to the known compound.

In another embodiment, the assay is a cell-free assay in which a MAGK protein or biologically active portion thereof is contacted with a test compound and the ability of the test compound to modulate (e.g., stimulate or inhibit) the activity of the MAGK protein or biologically active portion thereof is determined. Determining the ability of the test compound to modulate the activity of a MAGK protein can be accomplished, for example, by determining the ability of the MAGK protein to bind to a MAGK target molecule by one of the methods described above for determining direct binding. Determining the ability of the MAGK protein to bind to a MAGK target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a MAGK protein can be accomplished by determining the ability of the MAGK protein to interact with and/or convert a MAGK substrate (e.g., to produce a specific metabolite). In another alternative embodiment, determining the ability of the test compound to modulate the activity of a MAGK protein can be accomplished by determining the ability of the MAGK protein to further modulate the activity of a downstream effector of a MAGK target molecule. For example, the activity of the effector molecule on an appropriate target can be determined or the binding of the effector to an appropriate target can be determined as previously described.

In yet another embodiment, the cell-free assay involves contacting a MAGK protein or biologically active portion thereof with a known compound which binds the MAGK protein to form an assay mixture, contacting the assay mixture with a test compound, and determining the ability of the test compound to interact with the MAGK protein, wherein determining the ability of the test compound to interact with the MAGK protein comprises determining the ability of the MAGK protein to preferentially bind to or catalyze the transfer of a hydride moiety to or from the target substrate.

In more than one embodiment of the above assay methods of the present invention, it may be desirable to immobilize either MAGK or its target molecule to facilitate separation of complexed from uncomplexed forms of one or both of the proteins, as well as to accommodate automation of the assay. Binding of a test compound to a MAGK protein, or interaction of a MAGK protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/MAGK fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or MAGK protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of MAGK binding or activity determined using standard techniques.

Other techniques for immobilizing proteins on matrices can also be used in the screening assays of the invention. For example, either a MAGK protein or a MAGK target molecule can be immobilized utilizing conjugation of biotin and streptavidin. Biotinylated MAGK protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with MAGK protein or target molecules but which do not interfere with binding of the MAGK protein to its target molecule can be derivatized to the wells of the plate, and unbound target or MAGK protein trapped in the wells by antibody conjugation. Methods for detecting such complexes, in addition to those described above for the GST-immobilized complexes, include immunodetection of complexes using antibodies reactive with the MAGK protein or target molecule, as well as enzyme-linked assays which rely on detecting an enzymatic activity associated with the MAGK protein or target molecule.

In another embodiment, modulators of MAGK expression are identified in a method wherein a cell is contacted with a candidate compound and the expression of MAGK mRNA or protein in the cell is determined. The level of expression of MAGK mRNA or protein in the presence of the candidate compound is compared to the level of expression of MAGK mRNA or protein in the absence of the candidate compound. The candidate compound can then be identified as a modulator of MAGK expression based on this comparison. For example, when expression of MAGK mRNA or protein is greater (statistically significantly greater) in the presence of the candidate compound than in its absence, the candidate compound is identified as a stimulator of MAGK mRNA or protein expression. Alternatively, when expression of MAGK mRNA or protein is less (statistically significantly less) in the presence of the candidate compound than in its absence, the candidate compound is identified as an inhibitor of MAGK mRNA or protein expression. The level of MAGK mRNA or protein expression in the cells can be determined by methods described herein for detecting MAGK mRNA or protein.

In yet another aspect of the invention, the MAGK proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with MAGK (“MAGK-binding proteins” or “MAGK-6-bp”) and are involved in MAGK activity. Such MAGK-binding proteins are also likely to be involved in the propagation of signals by the MAGK proteins or MAGK targets as, for example, downstream elements of a MAGK-mediated signaling pathway. Alternatively, such MAGK-binding proteins are likely to be MAGK inhibitors.

The two-hybrid system is based on the modular nature of most transcription factors, which consist of separable DNA-binding and activation domains. Briefly, the assay utilizes two different DNA constructs. In one construct, the gene that codes for a MAGK protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a MAGK-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the MAGK protein.

In another aspect, the invention pertains to a combination of two or more of the assays described herein. For example, a modulating agent can be identified using a cell-based or a cell free assay, and the ability of the agent to modulate the activity of a MAGK protein can be confirmed in vivo, e.g., in an animal such as an animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a MAGK modulating agent, an antisense MAGK nucleic acid molecule, a ribozyme, a MAGK-specific antibody, or a MAGK-binding partner) can be used in an animal model to determine the efficacy, toxicity, or side effects of treatment with such an agent. Alternatively, an agent identified as described herein can be used in an animal model to determine the mechanism of action of such an agent. Examples of animal models of cancer include transplantable models (e.g., xenografts of colon tumors such as DLD-1, SW480, or SW620, or lung tumors into immunocompromised mice such as SCID or nude mice), transgenic models (e.g., B66-Min/+ mouse), chemical induction models, e.g., carcinogen (e.g., azoxymethan, 2-dimethylhydrazine, or N-nitrosodimethylamine) treated rats or mice, models of liver metastasis from colon cancer such as that described by Rashidi et al. (2000) Anticancer Res 20(2A):715, and cancer cell implantation or inoculation models as described in, for example, Fingert, et al. (1987) Cancer Res 46(14):3824-9 and Teraoka, et al. (1995) Jpn J Cancer Res 86(5):419-23.

Furthermore, this invention pertains to uses of novel agents identified by the above-described screening assays for treatments as described herein. In one embodiment, the invention features a method of treating a subject having a cellular growth or proliferation disease or disorder that involves administering to the subject a MAGK modulator such that treatment occurs. In another embodiment, the invention features a method of treating a subject having cancer, e.g. colon cancer or lung cancer, that involves treating a subject with a MAGK modulator, such that treatment occurs. Preferred MAGK modulators include, but are not limited to, MAGK proteins or biologically active fragments, MAGK nucleic acid molecules, MAGK antibodies, ribozymes, and MAGK antisense oligonucleotides designed based on the MAGK nucleotide sequences disclosed herein, as well as peptides, organic and non-organic small molecules identified as being capable of modulating MAGK expression and/or activity, for example, according to at least one of the screening assays described herein.

Any of the compounds, including but not limited to compounds such as those identified in the foregoing assay systems, may be tested for the ability to ameliorate cellular growth or proliferation disease or disorder symptoms. Cell-based and animal model-based assays for the identification of compounds exhibiting such an ability ameliorate cellular growth or proliferation disease or disorder systems are described herein.

In one aspect, cell-based systems, as described herein, may be used to identify compounds which may act to ameliorate cellular growth or proliferation disease or disorder symptoms. For example, such cell systems may be exposed to a compound, suspected of exhibiting an ability to ameliorate cellular growth or proliferation disease or disorder symptoms, at a sufficient concentration and for a time sufficient to elicit such an amelioration of cellular growth or proliferation disease or disorder symptoms in the exposed cells. After exposure, the cells are examined to determine whether one or more of the cellular growth or proliferation disease or disorder cellular phenotypes has been altered to resemble a more normal or more wild type, non-cellular growth or proliferation disease or disorder phenotype. Cellular phenotypes that are associated with cellular growth and/or proliferation disease states include aberrant proliferation, growth, and migration, anchorage independent growth, and loss of contact inhibition.

In addition, animal-based cellular growth or proliferation disease or disorder systems, such as those described herein, may be used to identify compounds capable of ameliorating cellular growth or proliferation disease or disorder symptoms. Such animal models may be used as test substrates for the identification of drugs, pharmaceuticals, therapies, and interventions which may be effective in treating cellular growth or proliferation disorders or diseases. For example, animal models may be exposed to a compound, suspected of exhibiting an ability to cellular growth or proliferation disease or disorder symptoms, at a sufficient concentration and for a time sufficient to elicit such an amelioration of cellular growth or proliferation disease or disorder symptoms in the exposed animals. The response of the animals to the exposure may be monitored by assessing the reversal of disorders or symptoms associated with cellular growth or proliferation disease, for example, reduction in tumor burden, tumor size, and invasive and/or metastatic potential before and after treatment.

With regard to intervention, any treatments which reverse any aspect of cellular growth or proliferation disease or disorder symptoms should be considered as candidates for human cellular growth or proliferation disease or disorder therapeutic intervention. Dosages of test agents may be determined by deriving dose-response curves.

Additionally, gene expression patterns may be utilized to assess the ability of a compound to ameliorate cellular growth and/or proliferation disease symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, cell growth, proliferation, differentiation, transformation, tumorigenesis, metastasis, and carcinogen exposure. Gene expression profiles may be generated, for example, by utilizing a differential display procedure, Northern analysis and/or RT-PCR. In one embodiment, MAGK gene sequences may be used as probes and/or PCR primers for the generation and corroboration of such gene expression profiles.

Gene expression profiles may be characterized for known states within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cellular growth or proliferation disease or disorder model system to more closely resemble the control system. Administration of a compound may, alternatively, cause the gene expression profile of a control system to begin to mimic a cellular growth and/or proliferation disease state. Such a compound may, for example, be used in further characterizing the compound of interest, or may be used in the generation of additional animal models.

B. Detection Assays

Portions or fragments of the cDNA sequences identified herein (and the corresponding complete gene sequences) and/or probes or primers based on the cDNA sequences identified herein, can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the MAGK nucleotide sequences, described herein, can be used to map the location of the MAGK genes on a chromosome. The mapping of the MAGK sequences to chromosomes is an important first step in correlating these sequences with genes associated with disease.

Briefly, MAGK genes can be mapped to chromosomes by preparing PCR primers (preferably 15-25 bp in length) from the MAGK nucleotide sequences. Computer analysis of the MAGK sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the MAGK sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the MAGK nucleotide sequences to design oligonucleotide primers, sublocalization can be achieved with panels of fragments from specific chromosomes. Other mapping strategies which can similarly be used to map a MAGK sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the MAGK gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The MAGK sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the MAGK nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The MAGK nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can comfortably provide positive individual identification with a panel of perhaps 10 to 1,000 primers which each yield a noncoding amplified sequence of 100 bases. If predicted coding sequences, such as that in SEQ ID NO:3, are used, a more appropriate number of primers for positive individual identification would be 500-2,000.

If a panel of reagents from MAGK nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of MAGK Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the MAGK nucleotide sequences or portions thereof, e.g., fragments derived from the noncoding regions of SEQ ID NO:1 having a length of at least 20 bases, preferably at least 30 bases.

The MAGK nucleotide sequences described herein can further be used to provide polynucleotide reagents, e.g., labeled or labelable probes which can be used in, for example, an in situ hybridization technique, to identify a specific tissue, e.g., thymus or brain tissue. This can be very useful in cases where a forensic pathologist is presented with a tissue of unknown origin. Panels of such MAGK probes can be used to identify tissue by species and/or by organ type.

In a similar fashion, these reagents, e.g., MAGK primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining MAGK protein and/or nucleic acid expression as well as MAGK activity, in the context of a biological sample (e.g., blood, serum, cells, tissue, e.g., lung or colon tissue or cells, or lung or colon tumor cells) to thereby determine whether an individual is afflicted with a disease or disorder, or is at risk of developing a disorder, associated with aberrant or unwanted MAGK expression or activity. The invention also provides for prognostic (or predictive) assays for determining whether an individual is at risk of developing a disorder associated with MAGK protein, nucleic acid expression or activity. For example, mutations in a MAGK gene can be assayed in a biological sample. Alternatively, the presence of MAGK expression and/or activity can be used in determining whether a person is at risk of having a tumor or at risk of having a tumor metastasize. Such assays can be used for prognostic or predictive purpose to thereby phophylactically treat an individual prior to the onset or progression of a disorder characterized by or associated with MAGK protein, nucleic acid expression or activity. In another example, the invention provides methods of determining the metastatic potential of a tumor.

Another aspect of the invention pertains to monitoring the influence of agents (e.g., drugs, compounds) on the expression or activity of MAGK in clinical trials.

These and other agents are described in further detail in the following sections.

1. Diagnostic Assays

The present invention encompasses methods for diagnostic and prognostic evaluation of cellular growth or proliferation disorders or diseases, e.g., cancer, including, but not limited to colon cancer and lung cancer, and for the identification of subjects exhibiting a predisposition to such conditions.

An exemplary method for detecting the presence or absence of MAGK protein or nucleic acid in a biological sample involves obtaining a biological sample from a test subject and contacting the biological sample with a compound or an agent capable of detecting MAGK protein or nucleic acid (e.g., mRNA, or genomic DNA) that encodes MAGK protein such that the presence of MAGK protein or nucleic acid is detected in the biological sample. A preferred agent for detecting MAGK mRNA or genomic DNA is a labeled nucleic acid probe capable of hybridizing to MAGK mRNA or genomic DNA. The nucleic acid probe can be, for example, the MAGK nucleic acid set forth in SEQ ID NO:1 or 3, or the DNA insert of the plasmid deposited with ATCC as Accession Number ______ , or a portion thereof, such as an oligonucleotide of at least 15, 30, 50, 100, 250 or 500 nucleotides in length and sufficient to specifically hybridize under stringent conditions to MAGK mRNA or genomic DNA. Other suitable probes for use in the diagnostic assays of the invention are described herein.

A preferred agent for detecting MAGK protein is an antibody capable of binding to MAGK protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect MAGK mRNA, protein, or genomic DNA in a biological sample in vitro as well as in vivo. For example, in vitro techniques for detection of MAGK mRNA include Northern hybridizations and in situ hybridizations. In vitro techniques for detection of MAGK protein include enzyme linked immunosorbent assays (ELISAs), Western blots, immunoprecipitations and immunofluorescence. In vitro techniques for detection of MAGK genomic DNA include Southern hybridizations. Furthermore, in vivo techniques for detection of MAGK protein include introducing into a subject a labeled anti-MAGK antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject. Also preferred are biological samples from tumors (e.g., tumor biopsies). Additional preferred biological samples include prostate tissue, liver tissue, breast tissue, skeletal muscle tissue, brain tissue, heart tissue, ovarian tissue, kidney tissue, lung tissue, vascular tissue, aortic tissue, thyroid tissue, placental tissue, intestinal tissue, cervical tissue, splenic tissue, esophageal tissue, thymic tissue, tonsillar tissue, lymph nodes and osteogenic cells. Particularly preferred samples are from colon tissue or lung tissue.

In another embodiment, the methods further involve obtaining a control biological sample from a control subject, contacting the control sample with a compound or agent capable of detecting MAGK protein, mRNA, or genomic DNA, such that the presence of MAGK protein, mRNA or genomic DNA is detected in the biological sample, and comparing the presence of MAGK protein, mRNA or genomic DNA in the control sample with the presence of MAGK protein, mRNA or genomic DNA in the test sample.

In one embodiment, increased levels of MAGK protein, mRNA or DNA (e.g., cDNA or genomic DNA) in the test sample as compared to the control sample is determinative or predictive of a MAGK-related aberrancy (e.g., a cellular growth or proliferation disease or disorder, for example, cancer). For example, 2-fold levels of expression of MAGK in the test sample as compared to the control sample may be determinative or predictive of a MAGK-related aberrancy. Preferably, 5-fold, 10-fold, 100-fold, 500-fold or 1000-fold levels of expression of MAGK in the test sample as compared to the control sample may be determinative or predictive of a MAGK-related aberrancy.

The invention also encompasses kits for detecting the presence of MAGK in a biological sample. For example, the kit can comprise a labeled compound or agent capable of detecting MAGK protein or mRNA in a biological sample; means for determining the amount of MAGK in the sample; and means for comparing the amount of MAGK in the sample with a standard. The compound or agent can be packaged in a suitable container. The kit can further comprise instructions for using the kit to detect MAGK protein or nucleic acid.

2. Prognostic Assays

The diagnostic methods described herein can furthermore be utilized to identify subjects having or at risk of developing a disease or disorder associated with aberrant or unwanted MAGK expression or activity. As used herein, the term “aberrant” includes a MAGK expression or activity which deviates from the wild type MAGK expression or activity. Aberrant expression or activity includes increased or decreased expression or activity, as well as expression or activity which does not follow the wild type developmental pattern of expression or the subcellular pattern of expression. For example, aberrant MAGK expression or activity is intended to include the cases in which a mutation in the MAGK gene causes the MAGK gene to be under-expressed or over-expressed and situations in which such mutations result in a non-functional MAGK protein or a protein which does not function in a wild-type fashion, e.g., a protein which does not interact with a MAGK substrate, or one which interacts with a non-MAGK substrate. As used herein, the term “unwanted” includes an unwanted phenomenon involved in a biological response such as cellular proliferation. For example, the term unwanted includes a MAGK expression or activity which is undesirable in a subject.

The assays described herein, such as the preceding diagnostic assays or the following assays, can be utilized to identify a subject having or at risk of developing a disorder associated with a misregulation in MAGK protein activity or nucleic acid expression, such as a cellular growth or proliferation disease or disorder (e.g., cancer, including, but not limited to, lung or colon cancer). Alternatively, the prognostic assays can be utilized to identify a subject having or at risk for developing a disorder associated with a misregulation in MAGK protein activity or nucleic acid expression, such as, a cellular growth or proliferation disease or disorder. Thus, the present invention provides a method for identifying a disease or disorder associated with aberrant or unwanted MAGK expression or activity in which a test sample is obtained from a subject and MAGK protein or nucleic acid (e.g., mRNA or genomic DNA) is detected, wherein the presence of MAGK protein or nucleic acid is diagnostic for a subject having or at risk of developing a disease or disorder associated with aberrant or unwanted MAGK expression or activity. As used herein, a “test sample” refers to a biological sample obtained from a subject of interest. For example, a test sample can be a biological fluid (e.g., serum), cell sample, e.g., a colon or lung epithelial cell sample, or tissue, e.g., colon or lung tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted MAGK expression or activity. For example, such methods can be used to determine whether a subject can be effectively treated with an agent for a cellular growth or proliferation disease or disorder. Thus, the present invention provides methods for determining whether a subject can be effectively treated with an agent for a disorder associated with aberrant or unwanted MAGK expression or activity in which a test sample is obtained and MAGK protein or nucleic acid expression or activity is detected (e.g., wherein the abundance of MAGK protein or nucleic acid expression or activity is diagnostic for a subject that can be administered the agent to treat a disorder associated with aberrant or unwanted MAGK expression or activity).

The methods of the invention can also be used to detect genetic alterations in a MAGK gene, thereby determining if a subject with the altered gene is at risk for a disorder characterized by misregulation in MAGK protein activity or nucleic acid expression, such as a cellular growth or proliferation disease or disorder. In preferred embodiments, the methods include detecting, in a sample of cells from the subject, the presence or absence of a genetic alteration characterized by at least one of an alteration affecting the integrity of a gene encoding a MAGK-protein, or the mis-expression of the MAGK gene. For example, such genetic alterations can be detected by ascertaining the existence of at least one of 1) a deletion of one or more nucleotides from a MAGK gene; 2) an addition of one or more nucleotides to a MAGK gene; 3) a substitution of one or more nucleotides of a MAGK gene, 4) a chromosomal rearrangement of a MAGK gene; 5) an alteration in the level of a messenger RNA transcript of a MAGK gene, 6) aberrant modification of a MAGK gene, such as of the methylation pattern of the genomic DNA, 7) the presence of a non-wild type splicing pattern of a messenger RNA transcript of a MAGK gene, 8) a non-wild type level of a MAGK-protein, 9) allelic loss of a MAGK gene, and 10) inappropriate post-translational modification of a MAGK-protein. As described herein, there are a large number of assays known in the art which can be used for detecting alterations in a MAGK gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a MAGK gene (see Abravaya et al. (1995) Nucleic Acids Res.23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a MAGK gene under conditions such that hybridization and amplification of the MAGK gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a MAGK gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in MAGK can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in MAGK can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MAGK gene and detect mutations by comparing the sequence of the sample MAGK with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the MAGK gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type MAGK sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in MAGK cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a MAGK sequence, e.g., a wild-type MAGK sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in MAGK genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control MAGK nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a MAGK gene.

Furthermore, any cell type or tissue in which MAGK is expressed (see Example 1) may be utilized in the prognostic assays described herein.

3. Monitoring of Effects During Clinical Trials

Monitoring the influence of agents (e.g., drugs) on the expression or activity of a MAGK protein (e.g., the modulation of cell proliferation, growth, and/or migration) can be applied not only in basic drug screening, but also in clinical trials. For example, the effectiveness of an agent determined by a screening assay as described herein to increase MAGK gene expression, protein levels, or upregulate MAGK activity, can be monitored in clinical trials of subjects exhibiting decreased MAGK gene expression, protein levels, or downregulated MAGK activity. Alternatively, the effectiveness of an agent determined by a screening assay to decrease MAGK gene expression, protein levels, or downregulate MAGK activity, can be monitored in clinical trials of subjects exhibiting increased MAGK gene expression, protein levels, or upregulated MAGK activity. In such clinical trials, the expression or activity of a MAGK gene, and preferably, other genes that have been implicated in, for example, a MAGK-associated disorder can be used as a “read out” or markers of the phenotype of a particular cell.

For example, and not by way of limitation, genes, including MAGK, that are modulated in cells by treatment with an agent (e.g., compound, drug or small molecule) which modulates MAGK activity (e.g., identified in a screening assay as described herein) can be identified. Thus, to study the effect of agents on MAGK-associated disorders (e.g., disorders characterized by deregulated cell proliferation and/or migration), for example, in a clinical trial, cells can be isolated and RNA prepared and analyzed for the levels of expression of MAGK and other genes implicated in the MAGK-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of MAGK or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a MAGK protein, mRNA, or genomic DNA in the preadministration sample; (iii) obtaining one or more post-administration samples from the subject; (iv) detecting the level of expression or activity of the MAGK protein, mRNA, or genomic DNA in the post-administration samples; (v) comparing the level of expression or activity of the MAGK protein, mRNA, or genomic DNA in the pre-administration sample with the MAGK protein, mRNA, or genomic DNA in the post administration sample or samples; and (vi) altering the administration of the agent to the subject accordingly. For example, increased administration of the agent may be desirable to increase the expression or activity of MAGK to higher levels than detected, i.e., to increase the effectiveness of the agent. Alternatively, decreased administration of the agent may be desirable to decrease expression or activity of MAGK to lower levels than detected, i.e. to decrease the effectiveness of the agent. According to such an embodiment, MAGK expression or activity may be used as an indicator of the effectiveness of an agent, even in the absence of an observable phenotypic response.

D. Methods of Treatment:

The present invention provides for both prophylactic and therapeutic methods of treating a subject at risk of (or susceptible to) a disorder or having a disorder associated with aberrant or unwanted MAGK expression or activity, e.g., a membrane-associated guanylate kinase-associated disorder such as a cellular growth or proliferation disorder, e.g., cancer, including, but not limited to, lung cancer and colon cancer. With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the MAGK molecules of the present invention or MAGK modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant or unwanted MAGK expression or activity, by administering to the subject an agent which modulates MAGK expression or at least one MAGK activity. Subjects at risk for a disease which is caused or contributed to by aberrant or unwanted MAGK expression or activity can be identified by, for example, any or a combination of diagnostic or prognostic assays as described herein. Administration of a prophylactic agent can occur prior to the manifestation of symptoms characteristic of the MAGK aberrancy, such that a disease or disorder is prevented or, alternatively, delayed in its progression. Depending on the type of MAGK aberrancy, for example, a MAGK, MAGK agonist or MAGK antagonist agent can be used for treating the subject. In a preferred embodiment, a MAGK antagonist (e.g., a MAGK inhibitor) can be used for treating a subject at risk for cancer. The appropriate agent can be determined based on screening assays described herein.

2. Therapeutic Methods

Another aspect of the invention pertains to methods of modulating MAGK expression or activity for therapeutic purposes. Accordingly, in an exemplary embodiment, the modulatory method of the invention involves contacting a cell with a MAGK or agent that modulates one or more of the activities of MAGK protein activity, e.g., modulation of cellular growth signaling, cellular growth or proliferation, associated with the cell (e.g., a colon or lung epithelial cell). An agent that modulates MAGK protein activity can be an agent as described herein, such as a nucleic acid or a protein, a naturally-occurring target molecule of a MAGK protein (e.g., a MAGK substrate), a MAGK antibody, a MAGK agonist or antagonist, a peptidomimetic of a MAGK agonist or antagonist, or other small molecule. In one embodiment, the agent stimulates one or more MAGK activities. Examples of such stimulatory agents include active MAGK protein and a nucleic acid molecule encoding MAGK that has been introduced into the cell. In another embodiment, the agent inhibits one or more MAGK activities. Examples of such inhibitory agents include antisense MAGK nucleic acid molecules, ribozymes, anti-MAGK antibodies, and MAGK inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a MAGK protein or nucleic acid molecule. In one embodiment, the method involves administering an agent (e.g., an agent identified by a screening assay described herein), or combination of agents that modulates (e.g., upregulates or downregulates) MAGK expression or activity. In another embodiment, the method involves administering a MAGK protein or nucleic acid molecule as therapy to compensate for reduced, aberrant, or unwanted MAGK expression or activity.

Stimulation of MAGK activity is desirable in situations in which MAGK is abnormally downregulated and/or in which increased MAGK activity is likely to have a beneficial effect. Likewise, inhibition of MAGK activity is desirable in situations in which MAGK is abnormally upregulated and/or in which decreased MAGK activity is likely to have a beneficial effect.

(i) Methods for Inhibiting Target Gene Expression, Synthesis, or Activity

As discussed above, genes involved in cellular growth or proliferation diseases or disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate cellular growth or proliferation disease or disorder symptoms. Such molecules may include, but are not limited to, small organic molecules, peptides, antibodies, and the like.

For example, compounds can be administered that compete with endogenous ligand for the MAGK protein. The resulting reduction in the amount of ligand-bound MAGK protein will modulate endothelial cell physiology. Compounds that can be particularly useful for this purpose include, for example, soluble proteins or peptides, such as peptides comprising one or more of the extracellular domains, or portions and/or analogs thereof, of the MAGK protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the MAGK receptor site, but do not activate the protein, (e.g., receptor-ligand antagonists) can be effective in inhibiting MAGK protein activity.

Further, antisense and ribozyme molecules, as described herein, which inhibit expression of the MAGK gene may also be used in accordance with the invention to inhibit aberrant MAGK gene activity. Still further, triple helix molecules may be utilized in inhibiting aberrant MAGK gene activity.

Antibodies that are both specific for the MAGK protein and interfere with its activity may also be used to modulate or inhibit MAGK protein function. Such antibodies may be generated using standard techniques described herein, against the MAGK protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

Any of the administration techniques described below which are appropriate for peptide administration may be utilized to effectively administer inhibitory target gene antibodies to their site of action.

(ii) Methods for Restoring or Enhancing Target Gene Activity

Genes that cause cellular growth or proliferation diseases or disorders may be underexpressed within cellular growth or proliferative situations. Alternatively, the activity of the protein products of such genes may be decreased, leading to the development of cellular growth or proliferation disease or disorder symptoms. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to cellular growth or proliferation disease or disorder conditions.

Described in this section are methods whereby the level MAGK activity may be increased to levels wherein cellular growth or proliferation disease or disorder symptoms are ameliorated. The level of MAGK activity may be increased, for example, by either increasing the level of MAGK gene expression or by increasing the level of active MAGK protein which is present.

For example, a MAGK protein, at a level sufficient to ameliorate cellular growth or proliferation disease or disorder symptoms may be administered to a patient exhibiting such symptoms. Any of the techniques discussed below may be used for such administration. One of skill in the art will readily be able to ascertain the concentration of effective, non-toxic doses of the MAGK protein, utilizing techniques such as those described above.

Additionally, RNA sequences encoding a MAGK protein may be directly administered to a patient exhibiting cellular growth or proliferation disease or disorder symptoms, at a concentration sufficient to produce a level of MAGK protein such that cellular growth or proliferation disease or disorder symptoms are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a MAGK gene, or a portion thereof, that directs the production of a normal MAGK protein with MAGK function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of MAGK gene sequences into human cells.

Cells, preferably, autologous cells, containing MAGK expressing gene sequences may then be introduced or reintroduced into the subject at positions which allow for the amelioration of cellular growth or proliferation disease or disorder symptoms. Such cell replacement techniques may be preferred, for example, when the gene product is a secreted, extracellular gene product.

3. Pharmacogenomics

The MAGK molecules of the present invention, as well as agents, or modulators which have a stimulatory or inhibitory effect on MAGK activity (e.g., MAGK gene expression) as identified by a screening assay described herein can be administered to individuals to treat (prophylactically or therapeutically) MAGK-associated disorders (e.g., cell growth or proliferative diseases or disorders), associated with aberrant or unwanted MAGK activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a MAGK molecule or MAGK modulator as well as tailoring the dosage and/or therapeutic regimen of treatment with a MAGK molecule or MAGK modulator.

Pharmacogenomics deals with clinically significant hereditary variations in the response to drugs due to altered drug disposition and abnormal action in affected persons. See, for example, Eichelbaum, M. et al. (1996) Clin. Exp.Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a MAGK protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a MAGK molecule or MAGK modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a MAGK molecule or MAGK modulator, such as a modulator identified by one of the exemplary screening assays described herein.

This invention is further illustrated by the following examples which should not be construed as limiting. The contents of all references, patents and published patent applications cited throughout this application, as well as the Figures, are incorporated herein by reference.

### EXAMPLES

### Example 1

### Identification and Characterization of Human MAGK cDNA

In this example, the identification and characterization of the gene encoding human MAGK (clone Fbh21910) also referred to herein as “21910” is described.

Isolation of the MAGK cDNA

The invention is based, at least in part, on the discovery of a human gene encoding a novel protein, referred to herein as MAGK. The entire sequence of human clone Fbh21910 was determined and found to contain an open reading frame termed human “MAGK”, set forth in FIG. 1. The amino acid sequence of the human MAGK expression product is set forth in FIG. 1. The MAGK protein sequence set forth in SEQ ID NO:2 comprises about 675 amino acids and is shown in FIGS. 1A-C. The coding region (open reading frame) of SEQ ID NO:1, is set forth as SEQ ID NO:3. Clone Fbh21910, comprising the coding region of human MAGK, was deposited with the American Type Culture Collection (ATCC®), 10801 University Boulevard, Manassas, Va. 20110-2209, on ______, and assigned Accession No. ______.

Analysis of the Human MAGK Molecule

The amino acid sequence of human MAGK was analyzed using the program PSORT (http://www.psort.nibb.ac.jp) to predict the localization of the protein within the cell. This program assesses the presence of different targeting and localization amino acid sequences within the query sequence. The results of the analysis predict that human MAGK (SEQ ID NO:2) is intracellular (e.g. nuclear, cytoplasmic, cytoskeletal).

A search of the amino acid sequence of MAGK was also performed against the ProSite database. This search resulted in the identification of a “ATP/GTP-binding site motif A (P-loop)” in the amino acid sequence of MAGK (SEQ ID NO:2) at about residues 404-411 and a “guanylate kinase signature” in the amino acid sequence of MAGK (SEQ ID NO:2) at about residues 514-531. This search also resulted in the identification in the amino acid sequence of human MAGK of a potential N-glycosylation site at about residues 82-85, a number of potential protein kinase C phosphorylation sites at about residues 84-86, 130-132, 253-255, 270-272, 432-434, 514-516, 517-519, 562-564, 569-571, 576-578, 581-583, and 584-586, a number of potential casein kinase II phosphorylation sites at about residues 14-17, 25-28, 97-100, 137-140, 143-146, 383-386, 422-425, 465-468, 517-520, 558-561 and 646-649, a tyrosine kinase phosphorylation site at about residues 586-593, a number of potential N-myristoylation sites at about residues 205-210, 247-2525, and 405-410, and a potential amidation site at about residues 72-76.

A search of the amino acid sequence of MAGK was also performed against the HMM database (FIGS. 3A-C). This search resulted in the identification of a “guanylate kinase domain” in the amino acid sequence of MAGK (SEQ ID NO:2) at about residues 515-624 (score=139.4), a “PDZ domain” in the amino acid sequence of MAGK (SEQ ID NO:2) at about residues 256-335 (score=52.4), a “SH3 domain” in the amino acid sequence of MAGK (SEQ ID NO:2) at about residues 348-415 (score=5.2).

Other HMM hits of interest are listed in FIGS. 3A-C, for example, a “NAD-dependent DNA ligase domain” at about residues 529-535 (score=2.3), an “X-Pro dipeptidyl-peptidase domain” at about residues 642-658 (score=−0.0), and a “caulimovirus movement protein domain” at about residues 420-673 (score=−184.0).

Tissue Distribution of MAGK by In situ Analysis

For in situ analysis, various tissues, e.g. tissues obtained from normal lung and colon and lung and colon tumors, were first frozen on dry ice. Ten-micrometer-thick sections of the tissues were post-fixed with 4% formaldehyde in DEPC treated 1×phosphate-buffered saline at room temperature for 10 minutes before being rinsed twice in DEPC 1×phosphate-buffered saline and once in 0.1 M triethanolamine-HCl(pH 8.0). Following incubation in 0.25% acetic anhydride-0.1 M triethanolamine-HCl for 10 minutes, sections were rinsed in DEPC 2×SSC (1×SSC is 0.15M NaCl plus 0.015M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with ³⁵S-radiolabeled (5×10⁷ cpm/ml) cRNA probes. Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1×Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2×SSC. Sections are then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides were then rinsed with 2×SSC at room temperature, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

In situ hybridization results indicated no expression in 2 normal lung samples. By contrast, expression was detected in 2 of 4 lung tumor samplels. Results further indicated no expression in 3 normal tumor samples and strong expression in 4 of 4 primary colon tumors tested and 3 of 3 colon metastases tested. Breast and ovary tissue also showed tumor specific expression.

Tissue Expression Analysis of MAGK mRNA Using Tagman Analysis

This example describes the tissue distribution of human MAGK mRNA (huMAGK) in a variety of cells and tissues, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., lung tumor samples, normal lung samples, colon tumor samples, and normal colon samples, and used as the starting material for PCR amplification. In addition to the 5′ and 3′ gene-specific primers, a gene-specific obligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyflourescein), or VIC) and a quencher dye (TAMRA (6-carboxy-N,N,N′,N′-tetramethylrhodamine) at the 3′ end of the probe.

During the PCR reaction, cleavage of the probe seperates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

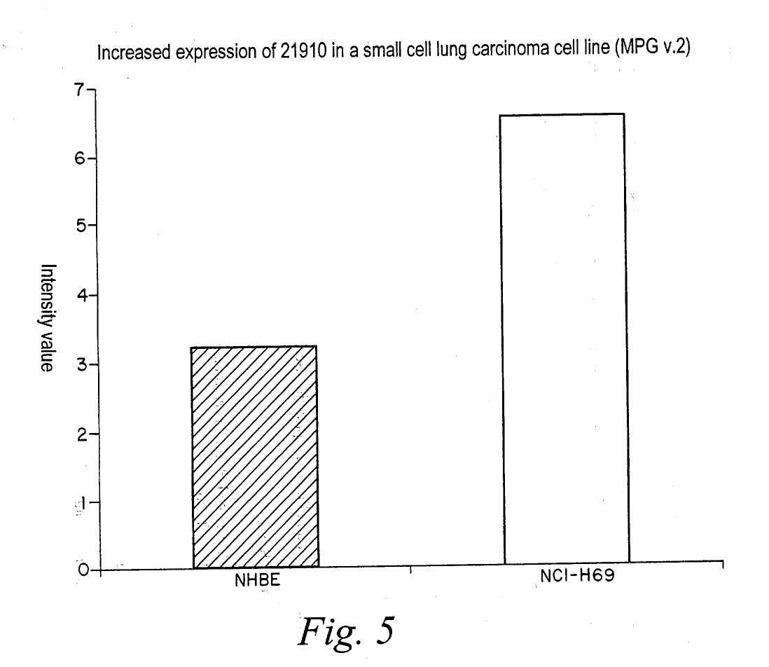

The expression levels of human MAGK mRNA in various human cell types and tissues was first determined in an array profiling experiment comparing the expression of genes in lung tumor cell lines versus normal bronchial epithelium. As shown in FIG. 5, MAGK expression is increased 2-fold in a small cell lung tumor line as compared to normal epithelium.

The RNA used in the array profiling experiment was isolated from the following cell lines: NHBE (available from Clonetics®) and NCI-H69 (available from ATCC®). NHBE cells were grown in BEGM (bronchial epithelium growth) Bulletkit® medium. The cells were grown to 80% confluency in a T175 flask and harvested for RNA by the Qiagen® Midi RNA preparation method. NCI-H69 cells were grown in suspension in T175 flasks in RPMI+2% Hyclone FBS, 2 mM L-Glutamine, 10 mM HEPES, and 1/100 Gibco® Selenium/Insulin/Transferrin supplement medium. RNA was prepared with the Qiagen® RNA Midi Kit, as directed by the manufacturer.

The expression levels of human MAGK mRNA in various human cell types and tissues were analyzed in detail in a second experiment using the Taqman procedure. As shown in Table 1, the highest MAGK expression was detected in brain, epithelial cells, and fetal heart.

TABLE 1
Expression of Human MAGK

| | Mean | Mean | |
|---|---|---|---|
| | huMAGK CT | Beta 2 CT | Normalized |
| Tissue Source | Value | Value | Expression |
| Aorta/normal | 35.91 | 24.30 | 0.52 |
| Fetal heart/normal | 27.07 | 20.91 | 22.72 |
| Heart/normal | 27.99 | 20.00 | 6.39 |
| Heart/CHF | 29.27 | 21.82 | 9.32 |
| Vein/normal | 30.94 | 20.60 | 1.25 |
| Spinal cord/normal | 27.43 | 20.11 | 10.17 |
| Brain cortex/normal | 26.85 | 22.17 | 63.15 |
| Brain hypothalamus | 26.48 | 21.08 | 38.47 |
| Glial cells (Astro) | 27.69 | 22.54 | 45.91 |
| Brain/Glioblastoma | 28.13 | 19.46 | 3.99 |
| Breast/normal | 29.03 | 20.52 | 4.47 |
| Breast tumor/IDC | 29.08 | 19.77 | 2.56 |
| OVARY/normal | 31.24 | 21.99 | 2.67 |
| OVARY/tumor | 29.61 | 20.44 | 2.82 |
| Pancreas | 32.24 | 25.20 | 12.34 |
| Prostate/normal | 28.34 | 20.32 | 6.26 |
| Prostate/tumor | 27.04 | 19.23 | 7.24 |
| Colon/normal | 27.83 | 19.13 | 3.91 |
| Colon/tumor | 26.83 | 19.82 | 12.60 |
| Colon/IBD | 29.96 | 19.39 | 1.05 |
| Kidney/normal | 28.14 | 21.61 | 17.58 |
| Liver/normal | 29.76 | 20.11 | 2.02 |
| Liver fibrosis | 30.51 | 21.19 | 2.54 |
| Fetal liver/normal | 30.62 | 22.42 | 5.54 |
| Lung/normal | 28.89 | 19.04 | 1.77 |
| Lung/tumor | 28.32 | 19.55 | 3.73 |
| Lung/COPD | 28.09 | 19.19 | 3.40 |
| Spleen/normal | 33.65 | 21.52 | 0.36 |
| Tonsil/normal | 30.00 | 19.09 | 0.85 |
| Lymphnode/normal | 30.47 | 19.71 | 0.94 |
| Thymus/normal | 28.29 | 20.49 | 7.26 |
| Epithelial Cells | 27.68 | 21.46 | 21.72 |
| Endothelial Cells | 30.77 | 22.01 | 3.73 |
| Skeletal Muscle | 29.17 | 21.74 | 9.42 |
| Fibroblasts (Dermal) | 30.38 | 20.04 | 1.26 |
| Skin/normal | 31.58 | 22.05 | 2.20 |
| Adipose/normal | 29.83 | 20.08 | 1.89 |
| Osteoblast (primary) | 29.21 | 21.17 | 6.19 |
| Osteoblasts (Undiff) | 28.89 | 20.09 | 3.64 |
| Osteoblasts (Diff.) | 28.59 | 19.16 | 2.36 |
| Osteoclasts | 30.91 | 18.58 | 0.32 |
| Aortic SMC Early | 28.86 | 21.39 | 9.16 |
| Aortic SMC Late | 31.23 | 24.20 | 12.47 |
| Shear HUVE C | 28.63 | 21.41 | 10.93 |
| Static HUVE C | 28.75 | 21.56 | 11.16 |
| Osteoclast (Undiff.) | 32.69 | 17.97 | 0.06 |

As shown in Table 2, increased expression of human MAGK was detected in 6 of 8 lung tumor samples (T) versus normal lung tissue samples (N). As shown in Table 3, increased expression of huMAGK was detected in 4 of 7 colon tumor samples (T) versus normal colon tissue samples (N).

TABLE 2
Human MAGK Expression in Clinical Lung Samples

| | Mean | Mean | |
|---|---|---|---|
| Tissue | huMAGK CT | Beta 2 CT | Normalized |
| Source | Value | Value | Expression |
| Lung N | 33.1 | 22.3 | 6.2 |
| Lung N | 29.3 | 19.1 | 9.3 |
| Lung N | 24.9 | 15.2 | 13.1 |
| Lung N | 26.9 | 16.4 | 7.3 |
| Lung T | 24.9 | 16.3 | 30.3 |
| Lung T | 25.7 | 17.5 | 37.2 |
| Lung T | 28.1 | 17.9 | 9.2 |
| Lung T | 26.6 | 17.2 | 16.3 |
| Lung T | 26.9 | 19.2 | 54.4 |
| Lung T | 27.8 | 19.3 | 29.5 |
| Lung T | 27.0 | 17.9 | 20.1 |
| Lung T | 26.4 | 18.0 | 31.7 |

TABLE 3
Human MAGK Expression in Clinical Colon Samples

| | Mean | Mean | |
|---|---|---|---|
| Tissue | huMAGK | Beta 2 CT | Normalized |
| Source | CT Value | Value | Expression |
| Colon N | 26.8 | 16.9 | 13.7 |
| Colon N | 30.4 | 21.0 | 18.6 |
| Colon N | 27.9 | 18.1 | 15.0 |
| Colon N | 25.7 | 16.8 | 27.7 |
| Colon T | 24.4 | 16.3 | 49.2 |
| Colon T | 24.3 | 17.3 | 102.6 |
| Colon T | 25.2 | 16.2 | 25.3 |
| Colon T | 26.3 | 17.1 | 21.4 |
| Colon T | 24.4 | 16.4 | 49.0 |
| Colon T | 32.0 | 23.6 | 37.7 |
| Colon T | 25.5 | 16.1 | 19.2 |
| Liver Met | 26.4 | 17.2 | 21.8 |
| Liver Met | 29.0 | 19.6 | 19.7 |
| Liver Met | 28.8 | 18.1 | 7.7 |
| Liver Met | 29.4 | 17.8 | 4.1 |
| Liver N | 28.9 | 17.4 | 4.3 |
| Liver N | 31.2 | 23.0 | 44.7 |

These data reveal a significant up-regulation of MAGK mRNA in colon and lung carcinomas. Given that the mRNA for MAGK is expressed in a variety of tumors, with significant up-regulation in carcinoma samples in comparison to normal samples, it is believed that inhibition of MAGK activity may inhibit tumor progression by inhibiting cell growth signaling and cellular growth and proliferation.

Tissue Distribution of MAGK mRNA by Northern Analysis

This example describes the determination of tissue distribution of MAGK mRNA, as determined by Northern analysis.

Northern blot hybridizations with the various RNA samples are performed under standard conditions and washed under stringent conditions, i.e., 0.2×SSC at 65° C. The DNA probe is radioactively labeled with ³²P-dCTP using the Prime-It kit (Stratagene, La Jolla, Calif.) according to the instructions of the supplier. Filters containing human mRNA (MultiTissue Northern I and MultiTissue Northern II from Clontech, Palo Alto, Calif.) are probed in ExpressHyb hybridization solution (Clontech) and washed at high stringency according to manufacturer's recommendations.

### Example 2

### Expression of Recombinant MAGK Protein in Bacterial Cells

In this example, MAGK is expressed as a recombinant glutathione-S-transferase (GST) fusion polypeptide in E. coli and the fusion polypeptide is isolated and characterized. Specifically, MAGK is fused to GST and this fusion polypeptide is expressed in E. coli, e.g, strain PEB199. Expression of the GST-MAGK fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 3

### Expression of Recombinant MAGK Protein in COS Cells

To express the MAGK gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire MAGK protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the MAGK DNA sequence is amplified by PCR using two primers. The 5′ primer contains the restriction site of interest followed by approximately twenty nucleotides of the MAGK coding sequence starting from the initiation codon; the 3′ end sequence contains complementary sequences to the other restriction site of interest, a translation stop codon, the HA tag or FLAG tag and the last 20 nucleotides of the MAGK coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the MAGK gene is inserted in the correct orientation. The ligation mixture is transformed into E. coli cells (strains HB101, DH5α, SURE, available from Stratagene Cloning Systems, LaJolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the MAGK-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the MAGK polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA-specific monoclonal antibody. Precipitated polypeptides are then analyzed by SDS-PAGE.

Alternatively, DNA containing the MAGK coding sequence is cloned directly into the polylinker of the pCDNA/Amp vector using the appropriate restriction sites. The resulting plasmid is transfected into COS cells in the manner described above, and the expression of the MAGK polypeptide is detected by radiolabelling and immunoprecipitation using a MAGK specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
(a) a nucleic acid molecule comprising the nucleotide sequence set forth in SEQ ID NO:1;
(b) a nucleic acid molecule comprising the nucleotide sequence set forth in SEQ ID NO:3;
(c) a nucleic acid molecule comprising the nucleotide sequence contained in the plasmid deposited with ATCC® as Accession Number ______ ;
(d) a nucleic acid molecule comprising a nucleotide sequence which is at least 92% identical to the nucleotide sequence of SEQ ID NO:1 or 3;
(e) a nucleic acid molecule comprising a fragment of at least 50 nucleotides of a nucleic acid comprising the nucleotide sequence of SEQ ID NO:1 or 3;
(f) a nucleic acid molecule which encodes a polypeptide comprising the amino acid sequence set forth in SEQ ID NO:2;
(g) a nucleic acid molecule which encodes a polypeptide comprising an amino acid sequence at least about 97% identical to the amino acid sequence of SEQ ID NO:2;
(h) a nucleic acid molecule which encodes a fragment of a polypeptide comprising the amino acid sequence of SEQ ID NO:2, wherein the fragment comprises at least 15 contiguous amino acid residues of the amino acid sequence of SEQ ID NO:2;
(i) a nucleic acid molecule which encodes a naturally occurring allelic variant of a polypeptide comprising the amino acid sequence set forth in SEQ ID NO:2;
(j) a nucleic acid molecule comprising a nucleotide sequence which is complementary to the nucleotide sequence of the nucleic acid molecule of any one of subparts (a) to (i); and
(k) a nucleic acid molecule which hybridizes to a complement of the nucleic acid molecule of any one of subparts (a) to (d), (f), (g) and (i) under stringent conditions.

2. An isolated nucleic acid molecule comprising the nucleic acid molecule of claim 1, and a nucleotide sequence encoding a heterologous polypeptide.

3. A vector comprising the nucleic acid molecule of claim 1.

4. The vector of claim 3, which is an expression vector.

5. A host cell transfected with the expression vector of claim 4.

6. A method of producing a polypeptide comprising culturing the host cell of claim 5 in an appropriate culture medium to, thereby, produce the polypeptide.

7. An isolated polypeptide selected from the group consisting of:
(a) a fragment of a polypeptide comprising the amino acid sequence of SEQ ID NO:2, wherein the fragment comprises at least 15 contiguous amino acids of SEQ ID NO:2;
(b) a naturally occurring allelic variant of a polypeptide comprising the amino acid sequence of SEQ ID NO:2, wherein the polypeptide is encoded by a nucleic acid molecule which hybridizes to a nucleic acid molecule consisting of SEQ ID NO:1 or 3 under stringent conditions;
(c) a polypeptide which is encoded by a nucleic acid molecule comprising a nucleotide sequence which is at least 92% identical to a nucleic acid comprising the nucleotide sequence of SEQ ID NO:1 or 3;
(d) a polypeptide comprising an amino acid sequence which is at least 97% identical to the amino acid sequence of SEQ ID NO:2; and
(e) a polypeptide comprising the amino acid sequence of SEQ ID NO:2.

8. The polypeptide of claim 7, further comprising heterologous amino acid sequences.

9. An antibody which selectively binds to a polypeptide of claim 7.

10. A method for detecting the presence of a polypeptide of claim 7 in a sample comprising:
(a) contacting the sample with a compound which selectively binds to the polypeptide; and
(b) determining whether the compound binds to the polypeptide in the sample to thereby detect the presence of a polypeptide of claim 7 in the sample.

11. The method of claim 10, wherein the compound which binds to the polypeptide is an antibody.

12. A kit comprising a compound which selectively binds to a polypeptide of claim 7 and instructions for use.

13. A method for detecting the presence of a nucleic acid molecule of claim 1 in a sample comprising:
(a) contacting the sample with a nucleic acid probe or primer which selectively hybridizes to the nucleic acid molecule; and
(b) determining whether the nucleic acid probe or primer binds to a nucleic acid molecule in the sample to thereby detect the presence of a nucleic acid molecule of claim 1 in the sample.

14. The method of claim 13, wherein the sample is isolated from a sample selected from the group consisting of colon tissue and lung tissue.

15. The method of claim 13, wherein the sample is a tumor sample.

16. A kit comprising a compound which selectively hybridizes to a nucleic acid molecule of claim 1 and instructions for use.

17. A method for identifying a compound which binds to or modulates the activity of a polypeptide of claim 7 comprising:
(a) contacting the polypeptide, or a cell expressing the polypeptide with a test compound; and
(b) determining whether the compound binds to or modulates the activity of the polypeptide, to thereby identify a compound which binds to or modulates the activity of the polypeptide.

18. A method for identifying a compound capable of treating a cellular growth or proliferation disease or disorder comprising assaying the ability of the compound or agent to modulate MAGK expression or activity, thereby identifying a compound capable of treating a cellular growth or proliferation disease or disorder.

19. A method for determining if a subject is at risk for a cellular growth or proliferation disease or disorder comprising detecting aberrant or abnormal MAGK expression or activity in a sample of tumor cells from the subject, thereby determining if a subject is at risk for a cellular growth or proliferation disease or disorder.

20. A method for identifying a subject suffering from a cellular growth or proliferation disease or disorder comprising obtaining a biological sample from the subject, and detecting in the sample aberrant or abnormal MAGK expression or activity, thereby identifying a subject suffering from a cellular growth or proliferation disease or disorder.

21. A method for treating a subject having a cellular growth or proliferation disease or disorder characterized by aberrant MAGK polypeptide activity or aberrant MAGK nucleic acid expression comprising administering to the subject a MAGK modulator, thereby treating said subject having a cellular growth or proliferation disease or disorder.

22. The method of any one of claims 18 to 21, wherein the disease or disorder is cancer.

23. The method of claim 22, wherein the disease or disorder is lung cancer or colon cancer.

24. The method of claim 21, wherein the MAGK modulator is selected from the group consisting of a small molecule, an antibody specific for MAGK, a MAGK polypeptide, a fragment of a MAGK polypeptide, a MAGK nucleic acid molecule, a fragment of a MAGK nucleic acid molecule, an antisense MAGK nucleic acid molecule, and a ribozyme.

25. The method of claim 24, wherein said MAGK modulator is administered in a pharmaceutically acceptable formulation.

26. The method of claim 24, wherein said MAGK modulator is administered using a gene therapy vector.

27. A method of regulating metastasis in an individual comprising administering to the individual a MAGK modulator such that metastasis is regulated.

28. A method of inhibiting tumor progression in an individual comprising administering to the individual a MAGK inhibitor such that tumor progression is inhibited.


SEQUENCE LISTING (3 sequences)

SEQ ID NO: 1 - DNA, 4281 bp, Homo sapiens
  CDS (452)..(2476): All occurrences of n = any nucleotide
agtaccccaaactttttactttcctactttgctaaactttccagtattgtatatcatttttcttagctttaaaattgaatagtatgtttcacatttttggaaagggactttggtggggaggggggaatcttgatttcctcaccattatctgtaaagtgaaagtacttaatctacttaacaaaataatattacatacttatgattttctacgttgtatggtgattagaagtgggctctgtaaaaacctgctttgatattgtgattcttacacttttgtgccccccatctttccccttagatttccttcatggatactttaccatagcattattatgtgatgtgagaagtttttccctctgaagtaacatggatttkatactacagaatcaagagaattggcttataggaaaaattgatttataaaaagtggtacaggttttcatagataaccatgacaacatcccatatgaatgggcatgttacagaggaatcagacagcgaagtaaaaaatgttgatcttgcatcaccagaggaacatcagaagcaccgagagatggctgttgactgccctggagatttgggcaccaggatgatgccaatacgtcgaagtgcacagttggagcgtattcggcaacaacaggaggacatgaggcgtaggagagaggaagaagggaaaaagcaagaacttgaccttaattcttccatgagacttaagaaactagcccaaattcctccaaagaccggaatagataaccctatgtttgatacagaggaaggaattgtcttagaaagtcctcattatgctgtgaaaatattagaaatagaagacttgttttcttcacttaaacatatccaacatactttggtagattctcagagccaggaggatatttcactgcttttacaacttgttcaaaataaggatttccagaatgcatttaagatacacaatgccatcacagtacacatgaacaaggccagtcctccatttcctcttatctccaacgcacaagatcttgctcaagaggtacaaactgttttgaagccagttcatcataaggaaggacaagaactaactgctttgctgaatactccacatattcaggcacttttactggcccacgataaggttgctgagcaggaaatgcagctagagcccattacagatgagagagtttatgaaagtattggccagtatggaggagaaactgtaaaaatagttcgtatagaaaaggctcgtgatattccgttgggtgctacagttcgtaatgaaatggactctgtcatcattagccggatagtaaaagggggtgctgcagagaaaagtggtctgttgcatgaaggagatgaagttctagagattaatggcattgaaattcgggggaaagatgtcaatgaggtttttgacttgttgtctgatatgcatggtactttgacttttgtcctgattcccagtcaacagatcaagccgcctcctgccaaggaaacagtaatccatgtaaaagctcattttgactatgacccctcagatgacccttatgttccatgtcgagagttaggtctgtcttttcaaaaaggtgatatacttcatgtgatcagtcaagaagatccaaactggtggcaggcctacagggaaggggacgaagataatcaacctctagccgggcttgttccagggaaaagctttcagcagcaaagggaagccatgaaacaaaccatagaagaagataaggagccagaaaaatcaggaaaactgtggtgtgcaaagaagaataaaaagaagaggaaaaaggttttatataatgccaataaaaatgatgattatgacaacgaggagatcttaacctatgaggaaatgtcactttatcatcagccagcaaataggaagagacctatcatcttgattggtccacagaactgtggccagaatgaattgcgtcagaggctcatgaacaaagaaaaggaccgctttgcatctgcagttcctcatacaacccggagtaggcgagaccaagaagtagccggtagagattaccactttgtttcgcggcaagcattcgaggcagacatagcagctggaaagttcattgagcatggtgaatttgagaagaatttgtatggaactagcatagattctgtacggcaagtgatcaactctggcaaaatatgtcttttaagtcttcgtacacagtcattgaagactctccggaattcagatttgaaaccatatattatcttcattgcacccccttcacaagaaagacttcgggcattattggccaaagaaggcaagaatccaaagcctgaagagttgagagaaatcattgagaagacaagagagatggagcagaacaatggccactactttgatacggcaattgtgaattccgatcttgataaagcctatcaggaattgcttaggttaattaacaaacttgatactgaacctcagtgggtaccatccacttggctgaggtgaaagaaacatccattctgtggcatgttggacttgatctggcaaaaactgccaataggaggactgcccgacactgcagcaagattgaggataagatggaaggcagcagtataagctgtagatctgttcttagatctcttgaattagtgagacgacagttcccttaggcagtttgtgcatggcatcctttattctctatacatggctttagcggttcttgcctcattttgggattctaaatggaagctttcaacagagcattccattttgtcctgttaaaaccttttgttttcacctaaaccctttctgcttagttgtatctctgtgaaaaacttgtatacacaagcgtccatgtctcacacaaatattgatgtgattattcttaagtgttaaatcattaacacttaaatgacttcattgggaatattgagcagagggactgtgcttctatgcactgggcaaggcagtatttgcttaggaaactaatttagtcatcagagatactttcctaaaaaggaaaaataaaaaacaaaatggtgccactttgggttgaagctactttgttaggcttgaattcatttatatgtcttttgattcttaaaaaaacaaaaaacattccattagaagcaccagtttttttgctcagactttgtggatcagactctacactcaacacactctaatctacttaaaggtatacaaaatatgctgatcttttttaaattatgatttcctgaatttttttcttaagtcgtctcaactgatttactcacttagcttcccttccctcatcagcatagtataatagaatgtatgttacatttttatgaatggcaggtgttcattataatctgtattgacttaaaaagtttcttcctcatgatgctaatagttttttgtatacatgggaggatagcacatttgacagtttttgcatttttatgtatgagcacagtatcctatgactgtgctacgtatatataggtaataaactggaattctgttgatgaatatagctgctgtactgtatattaatatttaatagatcaacaaatggtcattgaaaacacttgtttagcattagaataaaattatatatgtccttgggaaatattatgacagttgactttaagatcaaaaggaaaggaagacctgaaagtcatttgaacattttaggaaaagaatattggagagaaaaaggtattaaatatatagaaataggtttttaacctaacaaggtctgcctcttatgacgagaatgcaacagcttggtaaatcataaaagaaacatttaagctaataggattttcgtactgtctctatagctgtagctttaaaattcaacgtatataattggcatggaaacttaatttgcagtcttttcaagcctttaggatagtgtgatgtgtaacaaacaacctcaaatgtgaatgccttgattttatttttatggtgactttagctacagcatttcctatacccagagctaaacactggaataatactgacatcatttaatttaacataagcaattatgtttaaggagtaatttgtgtcatgtacatatttgattgattttttttcttctacataattttatttgaacaaatgtagacagtttatgtcgcctttttctgttcaaatttgcatggcctatnaagttggctggagagtgttttatgtggnaatattttcaagataatgttccttangaagnaaataacattnttgggttgagggaaggaatgccatacactactgtctcttcagatctgaaatactccagtttagagccaggaaatttc

SEQ ID NO: 2 - protein, 675 aa, Homo sapiens
MTTSHMNGHVTEESDSEVKNVDLASPEEHQKHREMAVDCPGDLGTRMMPIRRSAQLERIRQQQEDMRRRREEEGKKQELDLNSSMRLKKLAQIPPKTGIDNPMFDTEEGIVLESPHYAVKILEIEDLFSSLKHIQHTLVDSQSQEDISLLLQLVQNKDFQNAFKIHNAITVHMNKASPPFPLISNAQDLAQEVQTVLKPVHHKEGQELTALLNTPHIQALLLAHDKVAEQEMQLEPITDERVYESIGQYGGETVKIVRIEKARDIPLGATVRNEMDSVIISRIVKGGAAEKSGLLHEGDEVLEINGIEIRGKDVNEVFDLLSDMHGTLTFVLIPSQQIKPPPAKETVIHVKAHFDYDPSDDPYVPCRELGLSFQKGDILHVISQEDPNWWQAYREGDEDNQPLAGLVPGKSFQQQREAMKQTIEEDKEPEKSGKLWCAKKNKKKRKKVLYNANKNDDYDNEEILTYEEMSLYHQPANRKRPIILIGPQNCGQNELRQRLMNKEKDRFASAVPHTTRSRRDQEVAGRDYHFVSRQAFEADIAAGKFIEHGEFEKNLYGTSIDSVRQVINSGKICLLSLRTQSLKTLRNSDLKPYIIFIAPPSQERLRALLAKEGKNPKPEELREIIEKTREMEQNNGHYFDTAIVNSDLDKAYQELLRLINKLDTEPQWVPSTWLR

SEQ ID NO: 3 - DNA, 2025 bp, Homo sapiens
  CDS (1)..(2025)
atgacaacatcccatatgaatgggcatgttacagaggaatcagacagcgaagtaaaaaatgttgatcttgcatcaccagaggaacatcagaagcaccgagagatggctgttgactgccctggagatttgggcaccaggatgatgccaatacgtcgaagtgcacagttggagcgtattcggcaacaacaggaggacatgaggcgtaggagagaggaagaagggaaaaagcaagaacttgaccttaattcttccatgagacttaagaaactagcccaaattcctccaaagaccggaatagataaccctatgtttgatacagaggaaggaattgtcttagaaagtcctcattatgctgtgaaaatattagaaatagaagacttgttttcttcacttaaacatatccaacatactttggtagattctcagagccaggaggatatttcactgcttttacaacttgttcaaaataaggatttccagaatgcatttaagatacacaatgccatcacagtacacatgaacaaggccagtcctccatttcctcttatctccaacgcacaagatcttgctcaagaggtacaaactgttttgaagccagttcatcataaggaaggacaagaactaactgctttgctgaatactccacatattcaggcacttttactggcccacgataaggttgctgagcaggaaatgcagctagagcccattacagatgagagagtttatgaaagtattggccagtatggaggagaaactgtaaaaatagttcgtatagaaaaggctcgtgatattccgttgggtgctacagttcgtaatgaaatggactctgtcatcattagccggatagtaaaagggggtgctgcagagaaaagtggtctgttgcatgaaggagatgaagttctagagattaatggcattgaaattcgggggaaagatgtcaatgaggtttttgacttgttgtctgatatgcatggtactttgacttttgtcctgattcccagtcaacagatcaagccgcctcctgccaaggaaacagtaatccatgtaaaagctcattttgactatgacccctcagatgacccttatgttccatgtcgagagttaggtctgtcttttcaaaaaggtgatatacttcatgtgatcagtcaagaagatccaaactggtggcaggcctacagggaaggggacgaagataatcaacctctagccgggcttgttccagggaaaagctttcagcagcaaagggaagccatgaaacaaaccatagaagaagataaggagccagaaaaatcaggaaaactgtggtgtgcaaagaagaataaaaagaagaggaaaaaggttttatataatgccaataaaaatgatgattatgacaacgaggagatcttaacctatgaggaaatgtcactttatcatcagccagcaaataggaagagacctatcatcttgattggtccacagaactgtggccagaatgaattgcgtcagaggctcatgaacaaagaaaaggaccgctttgcatctgcagttcctcatacaacccggagtaggcgagaccaagaagtagccggtagagattaccactttgtttcgcggcaagcattcgaggcagacatagcagctggaaagttcattgagcatggtgaatttgagaagaatttgtatggaactagcatagattctgtacggcaagtgatcaactctggcaaaatatgtcttttaagtcttcgtacacagtcattgaagactctccggaattcagatttgaaaccatatattatcttcattgcacccccttcacaagaaagacttcgggcattattggccaaagaaggcaagaatccaaagcctgaagagttgagagaaatcattgagaagacaagagagatggagcagaacaatggccactactttgatacggcaattgtgaattccgatcttgataaagcctatcaggaattgcttaggttaattaacaaacttgatactgaacctcagtgggtaccatccacttggctgagg